SEQUENCE LISTING (12 sequences)

SEQ ID NO: 1 - DNA, 1542 bp, Homo sapiens
  CDS (158)..(1414)
ggtcgctttaagaaaggagtagctgtaatctgaagcctgctggacgctggattagaaggcagcaaaaaaagctctgtgctggctggagccccctcagtgtgcaggcttagagggactaggctgggtgtggagctgcagcgtatccacaggccccaggatgcaggccctggtgctactcctctgcattggagccctcctcgggcacagcagctgccagaaccctgccagccccccggaggagggctccccagaccccgacagcacaggggcgctggtggaggaggaggatcctttcttcaaagtccccgtgaacaagctggcagcggctgtctccaacttcggctatgacctgtaccgggtgcgatccagcacgagccccacgaccaacgtgctcctgtctcctctcagtgtggccacggccctctcggccctctcgctgggagcggagcagcgaacagaatccatcattcaccgggctctctactatgacttgatcagcagcccagacatccatggtacctataaggagctccttgacacggtcactgccccccagaagaacctcaagagtgcctcccggatcgtctttgagaagaagctgcgcataaaatccagctttgtggcacctctggaaaagtcatatgggaccaggcccagagtcctgacgggcaaccctcgcttggacctgcaagagatcaacaactgggtgcaggcgcagatgaaagggaagctcgccaggtccacaaaggaaattcccgatgagatcagcattctccttctcggtgtggcgcacttcaaggggcagtgggtaacaaagtttgactccagaaagacttccctcgaggatttctacttggatgaagagaggaccgtgagggtccccatgatgtcggaccctaaggctgttttacgctatggcttggattcagatctcagctgcaagattgcccagctgcccttgaccggaagcatgagtatcatcttcttcctgcccctgaaagtgacccagaatttgaccttgatagaggagagcctcacctccgagttcattcatgacatagaccgagaactgaagaccgtgcaggcggtcctcactgtccccaagctgaagctgagttatgaaggcgaagtcaccaagtccctgcaggagatgaagctgcaatccttgtttgattcaccagactttagcaagatcacaggcaaacccatcaagctgactcaggtggaacaccgggctggctttgagtggaacgaggatggggcgggaaccacccccagcccagggctgcagcctgcccacctcaccttcccgctggactatcaccttaaccagcctttcatcttcgtactgagggacacagacacaggggcccttctcttcattggcaagattctggaccccaggggcccctaatatcccagtttaatattccaataccctagaagaaaacccgagggacagcagattccacaggacacgaaggctgcccctgtaaggtttcaatgcatacaataaaagagctttatccctaacttctgtta

SEQ ID NO: 2 - protein, 418 aa, Homo sapiens
MQALVLLLCIGALLGHSSCQNPASPPEEGSPDPDSTGALVEEEDPFFKVPVNKLAAAVSNFGYDLYRVRSSTSPTTNVLLSPLSVATALSALSLGAEQRTESIIHRALYYDLISSPDIHGTYKELLDTVTAPQKNLKSASRIVFEKKLRIKSSFVAPLEKSYGTRPRVLTGNPRLDLQEINNWVQAQMKGKLARSTKEIPDEISILLLGVAHFKGQWVTKFDSRKTSLEDFYLDEERTVRVPMMSDPKAVLRYGLDSDLSCKIAQLPLTGSMSIIFFLPLKVTQNLTLIEESLTSEFIHDIDRELKTVQAVLTVPKLKLSYEGEVTKSLQEMKLQSLFDSPDFSKITGKPIKLTQVEHRAGFEWNEDGAGTTPSPGLQPAHLTFPLDYHLNQPFIFVLRDTDTGALLFIGKILDPRGP

SEQ ID NO: 3 - protein, 40 aa, Homo sapiens
  MISC_FEATURE (1)..(40): Residues 82-121 of human PEDF
PLSVATALSALSLGAEQRTESIIHRALYYDLISSPDIHGT

SEQ ID NO: 4 - DNA, 120 bp, Homo sapiens
cctctcagtgtggccacggccctctcggccctctcgctgggagcggagcagcgaacagaatccatcattcaccgggctctctactatgacttgatcagcagcccagacatccatggtacc

SEQ ID NO: 5 - protein, 20 aa, Homo sapiens
  MISC_FEATURE (1)..(20): Residues 76-95 of human PEDF
TNVLLSPLSVATALSALSLG

SEQ ID NO: 6 - DNA, 60 bp, Homo sapiens
accaacgtgctcctgtctcctctcagtgtggccacggccctctcggccctctcgctggga

SEQ ID NO: 7 - protein, 20 aa, Homo sapiens
  MISC_FEATURE (1)..(20): Residues 91-110 of human PEDF
ALSLGAEQRTESIIHRALYY

SEQ ID NO: 8 - DNA, 60 bp, Homo sapiens
gccctctcgctgggagcggagcagcgaacagaatccatcattcaccgggctctctactat

SEQ ID NO: 9 - protein, 20 aa, Homo sapiens
  MISC_FEATURE (1)..(20): Residues 106-125 of human PEDF
RALYYDLISSPDIHGTYKEL

SEQ ID NO: 10 - DNA, 60 bp, Homo sapiens
cgggctctctactatgacttgatcagcagcccagacatccatggtacctataaggagctc

SEQ ID NO: 11 - DNA, 1257 bp, Artificial
  chemically synthesized
atgcaagccctggtcctgctgctgtgcatcggggcgctgctgggtcacagctcatgtcaaaacccggccagtccgccggaagagggctcaccggacccggactcaacgggcgctctggttgaagaggaagatccgtttttcaaagttccagttaacaaactggcggcggccgtttcgaacttcgggtacgatctgtatcgcgtgcgctcttctatgagcccgaccacaaatgtgctgctgagcccgctgtcggtggcaaccgcgctgagcgctctgtcactgggcgcagatgaacgtactgaatccattattcatcgcgcgctgtattatgacctgattagctctccagacattcatggcacctacaaagaactgctggataccgtgactgcaccgcagaaaaacctgaaatcggcaagccgtatcgtatttgagaaaaaactgcgcattaaatcaagctttgttgctccactggagaaaagctatggtactcgcccgcgcgtgctgactggtaatccgcgcctggatctgcaagaaatcaacaattgggtgcaagctcagatgaaaggtaaactggcgcgcagcactaaagaaatcccagatgaaatcagtatcctgctgctgggcgtggcacactttaaaggccagtgggtcaccaaatttgatagccgtaaaacgagtctggaagatttttatctggatgaagagcgtactgtgcgcgttccaatgatgagtgacccgaaagcagttctgcgctacggtctggatagcgatctgagctgcaaaatcgctcaactgccgctgaccggctcaatgagcattattttctttctgccgctgaaagttacccaaaatctgaccctgatcgaagagagcctgacctccgaattcattcatgatatcgatcgcgaactgaaaacagttcaggcagtcctgaccgtcccgaaactgaaactgtcttatgaaggtgaagtgacaaaatcactgcaggaaatgaaactgcagagcctgttcgattccccggatttcagtaaaattaccggtaaaccgattaaactgacccaggtggaacatcgcgctggctttgaatggaacgaagacggggcaggtacgaccccgagcccaggcctgcaaccagcgcatctgacttttccactggattatcacctgaatcaaccgtttatctttgtgctgcgtgatactgacaccggcgctctgctgtttatcggtaaaatcctggacccgcgtggcccgtaa

SEQ ID NO: 12 - protein, 418 aa, Homo sapiens [sequence omitted]

(19) United States
(12) Patent Application Publication
(10) Pub. No.: US 2009/0069241 A1
(43) Pub. Date: Mar. 12, 2009

(54) Compositions and Methods for Use of Pigment Epithelial Derived Factor (PEDF) Peptide Fragments

(75) Inventors: Colin J Barnstable, Guilford, CT (US); Joyce Tombran-Tink, Parkville, MO (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignees: Yale University, New Haven, CT (US); The Curators of the University of Missouri, Kansas City, MO (US)

(21) Appl. No.: 12/278,744

(22) PCT Filed: Feb. 15, 2007
(86) PCT No.: PCT/US07/04109

§ 371 Date: Aug. 7, 2008

Related U.S. Application Data
(60) Provisional application No. 60/773,573, filed on Feb. 15, 2006.
(60) Provisional application No. 60/779,543, filed on Mar. 6, 2006.

(51) Int. Cl. A61K 38/00; C07K 14/00; C12N 15/11; C12N 1/21; C12P 21/04; A61P 25/00; A61K 31/7088; C12N 15/00; C07K 7/00

(52) U.S. Cl. 514/12; 530/326; 530/324; 514/13; 536/23.1; 435/320.1; 435/252.3; 536/23.4; 514/44; 435/69.1

(57) ABSTRACT

The invention provides PEDF peptides which retain the biological activity of full-length PEDF. Fusion proteins comprising a PEDF peptide are also provided. The invention further provides a codon-optimized PEDF coding sequence and method of expressing it in bacteria. Compositions, methods of use and kits are also provided.

### BACKGROUND OF THE INVENTION

Pigment derived-epithelium factor (PEDF) is a protein that is expressed in virtually all tissues of the human body, including nerve tissues (Tombran-Tink et al., 1989, Invest Opthalmol V is Sci. 30(8): 1700-1707; Tombran-Tink et al., 1991, Exp Eye Res. 53(3):411-414; Tombran-Tink et al., 1995, J Neurosci. 15(7 Pt 1):4992-5003). It is a member of the second group of the serpin family. PEDF is an angiogenesis inhibitor with neurotrophic properties (Dawson et al., 1999, Science 285:245-248; Tombran-Tink et al., 2003, Nat Rev Neurosci. 4:628-636; Crawford et al., 2001, J Cell Sci. 114(Pt 24):4421-4428; and Abe et al., 2004, Am J Pathol. 164:1225-1232). The protective effects of PEDF on cerebellar granule cells, hippocampal neurons, and spinal-cord motor neurons from increased glutamate excitotoxicity has been well documented (Taniwaki et al., 1995, J Neurochem. 64(6):2509-17; Bilak et al., 1999, J Neuropathol Exp Neurol. 58(7):719-728; DeCoster et al., 1999, J Neurosci Res. 56(6):604-610). The cell survival promoting actions of PEDF extend to neurons of the retina as well. PEDF prevents damage to rat rod photoreceptors exposed to H₂O₂ or constant bright light, to rods of the retinal degeneration (rd) and retinal degeneration slow (rds) mutant mice, and to Xenopus rods after RPE detachment. (Cao et al., 1999, J Neurosci Res. 57(6):789-800; Cayouette et al., 1999, Neurobiol Dis. 6(6):523-532; Cao et al., 2001, Invest Opthalmol V is Sci. 42(7):1646-1652; Jablonski et al., 2001, Glia 35(1):14-25). Reduced expression of PEDF plays a role in eye pathologies, including retinopathy and macular degeneration (Holekamp et al., 2002, Am J Opthalmol. 134:220-227). Increased expression of PEDF can induce regression of optical neovascularization (Mori et al., 2002, Invest Opthalmol V is Sci. 43:2428-34). PEDF expression controls tumor growth (Wang et al., 2003, Mol Ther. 2003; 8:72-79; Abe et al., 2004, Am J Pathol. 164:1225-1232). Thus, PEDF has many therapeutic applications in a variety of pathologies involving angiogenesis and/or neuronal degeneration.

Delivery of PEDF to the posterior segment of the eye has been achieved by injection of a single bolus of the native protein (Cao et al., 2001, Invest Opthalmol V is Sci. 42(7): 1646-1652; Ogata et al., 2001, Curr Eye Res. 22(4):245-252) or suitable titers of a virally-vectored PEDF gene directly into the vitreous cavity (Mori et al., 2002, Invest Opthalmol V is Sci. 43(6):1994-2000; Raisler et al., 2002, Proc Natl Acad Sci USA. 99(13):8909-8914; Gehlbach et al., 2003, Gene Ther. 10(8):637-646). These methods of delivery have resulted in therapeutic concentrations of PEDF at retinal target sites with minimal systemic side effects. Nevertheless, a single bolus injection of neuroprotective factors can provide only transient protection and multiple treatment may be required for long-term rescue, thus subjecting the recipient to the additional risk of injury and infection. While the use of virally-vectored gene shows great promise for long-term gene expression, injections of such vectors encoding therapeutically active genes can cause severe inflammatory responses to antigenic viral proteins. In addition these transgenes are often transiently expressed, not expressed at therapeutic levels, or overexpressed above physiologically beneficial doses.

Human PEDF is a 50 kiloDalton (kD) protein, which limits the effectiveness of some forms of delivery for therapeutic applications. The options for alternative delivery systems are limited for large polypeptides such as PEDF. One way in which this problem might be overcome is to identify fragments of PEDF that maintain bioactivity. Identification of a functional fragment of PEDF is therefore desirable. Structure-function analysis on PEDF has been pursued (Becerra, 1997, Adv Exp Med Biol. 425:223-37; Bilak et al., 2002, J Neurosci. 22:9378-9386; Filleur et al., 2005, Cancer Res. 65:5144-5251; Tombran-Tink et al., 2005, J Struc Biol. 151:130-150).

There is a need in the art for a molecule retaining the activity of full-length PEDF and smaller in size. The present invention addresses this need.

### BRIEF SUMMARY OF THE INVENTION

The invention provides a peptide consisting essentially of a sequence selected from the group consisting of residues 82 to 121 of a human pigment epithelium-derived factor (PEDF), residues 76 to 95 of a human PEDF, residues 91 to 110 of a human PEDF, residues 106 to 125 of a human PEDF and variants thereof. Isolated nucleic acids encoding a peptide of the invention are included in the invention, as are vectors comprising the isolated nucleic acid, and a cell comprising the vector. The invention also provides compositions comprising a peptide or an isolated nucleic acid of the invention.

In one embodiment, the peptide is encapsulated in a biocompatible delivery polymer. Exemplary biocompatible delivery polymers include a hydrogel, a polymer comprising gelatin, a polymer comprising collagen, a polymer comprising alginate, and a polymer comprising poly (lactide-co-glycolide) (PGLA). In a preferred embodiment, the biocompatible delivery polymer is PGLA. In one embodiment, the peptide is contained within a nanotube, a nanosphere or a microsphere.

Fusion proteins are also provided by the invention. In one embodiment, the fusion protein comprises a first peptide operably fused to a second peptide, wherein the first peptide consists essentially of a sequence selected from the group consisting of residues 82 to 121 of a human pigment epithelium-derived factor (PEDF), residues 76 to 95 of a human PEDF, residues 91 to 110 of a human PED, residues 106 to 125 of a human PEDF F and variants thereof, and the second peptide is not a PEDF peptide. In one embodiment, the second peptide is selected from the group consisting of His6, FLAG-tag, Myc, LacZ, a cellular targeting sequence and a therapeutic polypeptide.

In another embodiment, the fusion protein comprises a first peptide operably fused to a second peptide, wherein the first peptide consists essentially of a sequence selected from the group consisting of residues 82 to 121 of a human pigment epithelium-derived factor (PEDF), residues 76 to 95 of a human PEDF, residues 91 to 110 of a human PEDF, residues 106 to 125 of a human PEDF and variants thereof, and the second peptide is a PEDF peptide that consists essentially of a sequence selected from the group consisting of residues 82 to 121 of a human pigment epithelium-derived factor (PEDF), residues 76 to 95 of a human PEDF, residues 91 to 110 of a human PEDF, residues 106 to 125 of a human PEDF and variants thereof, wherein the first and second peptides are not identical to each other.

In one embodiment, the fusion peptide is encapsulated in a biocompatible delivery polymer. In one embodiment, the peptide is contained within a nanotube, a nanosphere or a microsphere. The invention also includes an isolated nucleic acid encoding a fusion protein of the invention, as well as vectors comprising the isolated nucleic acid and a cell comprising the vector.

Compositions comprising a fusion protein or an isolated nucleic acid encoding a fusion protein of the invention are provided by the invention.

Kits comprising a peptide, a fusion protein, an isolated nucleic acid or a composition of the invention and an instructional material are also provided.

Further provided are a methods of using a peptide, fusion protein, isolated nucleic acid, or composition of the invention. In one embodiment, a method of treating a neurodegenerative disease in a mammal is provided, the method comprising administering to said mammal a therapeutically effective amount of a peptide consisting essentially of a sequence selected from the group consisting of residues 82 to 121 of a human pigment epithelium-derived factor (PEDF), residues 76 to 95 of a human PEDF, residues 91 to 110 of a human PEDF, residues 106 to 125 of a human PEDF and variants thereof. Preferably, the mammal is a human. In one embodiment, the neurodegenerative disease is selected from the group consisting of Parkinson's disease, Huntington's disease, Alzheimer's disease, diabetic retinopathy, and an ocular disease. In one embodiment, the ocular disease is selected from the group consisting of: ischemic injury, diabetic retinopathy, retinopathy of prematurity, macular degeneration and glaucoma.

In another embodiment, a method of treating a neurodegenerative disease in a mammal is provided, comprising administering a fusion protein of the invention. In yet another embodiment, a method of treating a neurodegenerative disease is provided, comprising administering an isolated nucleic acid encoding a peptide or fusion protein of the invention.

The invention further provides a codon-optimized coding sequence for a human PEDF. In one embodiment, the sequence comprises SEQ ID NO: 11 or variants thereof. A vector comprising SEQ ID NO: 11 or a variant thereof is provided. In one embodiment, the vector further comprise one or more of a thioredoxin coding sequence and a His₆ coding sequence operably linked to SEQ ID NO: 11 or variants thereof.

A method of preparing human PEDF in a bacterial cell is provided. The method includes the steps of a) introducing an expression cassette comprising SEQ ID NO: 11 or variants thereof into a bacterium; b) expressing said expression cassette to produce a human PEDF protein; and c) purifying said human PEDF protein.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIGS. 1A-1C are a series of representative images and graphs depicting the time-course of retina damage after 60 minutes ischemic injury. ONL: outer nuclear layer. INL: inner nuclear layer. IPL: inner plexiform layer. GCL: ganglion cell layer. The ganglion cell layer comprises retinal ganglion cells (RGCs) as well as other cells. FIG. 1A depicts images of hematoxylin and eosin-stained retinas at different times after ischemic injury. Calibration, 20 μm. FIG. 1B is a graph of the mean thickness of INL and IPL in retinas at different times after ischemic injury. FIG. 1C is a graph of the total surviving cells in the retinal ganglion cell layer at different times after ischemic injury. Morphometric analysis: 4×250 μm zones were sampled from all quadrant of the retina. The average measurement of these 4 zones was used for statistic analysis. All ischemic groups are compared with the control group. P values are from one-way ANOVA measurement (**p<0.01, ***p<0.001).

FIG. 2 is a series of images of retina after transient ischemia and stained using the TUNEL assay. The TUNEL assay was used to determine the spatial and temporal pattern of apoptosis in the retina after transient ischemia. Animals were allowed to recover for 6 hours (6 hr), 24 hours (24 hr), 48 hours (48 hr) or 7 days (7 d) post injury. ONL: outer nuclear layer. INL: inner nuclear layer. RGC: retinal ganglion cell layer. Bar=20 μm.

FIGS. 3A and 3B are a series of images and a graph illustrating survival of RGC layer cells after transient ischemic injury to the retina. FIG. 3A is a series of images of hematoxylin and eosin-stained retinas at various times after transient ischemia. ONL: outer nuclear layer. INL: inner nuclear layer. RGC: retinal ganglion cell layer. Bar=20 μm. FIG. 3B is a graph of morphometric analysis revealing the decrease in surviving cells in RGC layers after transient ischemia. Morphometric analysis of retinas shows the surviving cells in RGC layer decreased significantly in ischemic groups at all time points when compared with the uninjured normal retina group. P values are from one-way ANOVA measurement. **p is versus the uninjured normal retina group. (**p<0.01).

FIG. 4 is a series of images and graphs illustrating survival of RGC layer cells after transient ischemia, followed by intraocular injection of PBS (control) or full-length PEDF protein. Images at the top are hematoxylin and eosin-stained retinas at 48 hours (48 hr) and 7 days (7 d) after transient ischemia. ONL: outer nuclear layer. INL: inner nuclear layer. RGC: retinal ganglion cell layer. Bar=20 μm. Graphs at the bottom depict total surviving cells in the retinal ganglion cell layer. P values are from one-way ANOVA measurement compared with PBS injected group at the same time point. (**p<0.01; ***p<0.001).

FIGS. 5A-5E are a series of representative images and graphs of data exhibiting the morphologic and morphometric change of inner retina of animals subjected to ischemic injury and administration of different compounds. Animals were immediately intravitreous injected PBS, control peptide (PEDF₁₃₆₋₁₅₅), PEDF₈₂₋₁₂₁ peptide or PEDF protein after ischemic injury and allowed to recover for 48 hours (48 hr) or 7 days (7 d). FIG. 5A depicts representative images of hematoxylin and eosin-stained of retinas. Calibration, 20 μm. FIGS. 5B and 5D are graphs of the mean thickness of INL and IPL in retinas 48 hours and 7 days, respectively, after ischemic injury. FIGS. 5C and 5E are graphs of the total surviving cells in the retinal ganglion cell layer at 48 hours and 7 days, respectively. One-way ANOVA measurement was used for statistic analysis, *p values were versus the PBS-injected group; #p values were versus the control peptide group. (*p<0.05, **p<0.01, ***p<0.001, ##p<0.01).

FIGS. 6A and 6B are a series of image and a graph illustrating survival of RGC layer cells after transient ischemia, followed by intraocular injection of various compounds. PBS, PEDF₈₂₋₁₂₁ peptide or PEDF₁₃₆₋₁₅₅ peptide. FIG. 6A comprises images of hematoxylin and eosin-stained retinas 48 hours after transient ischemia and injection of either PBS or PEDF₁₃₆₋₁₅₅. Bar=20 μm. The graph on the right depict total surviving cells in the retinal ganglion cell layer. FIG. 6B comprises images of hematoxylin and eosin-stained retinas 48 hours and 7 days after transient ischemia and injection of either PBS or PEDF₈₂₋₁₂₁. Bar=20 μm. The graphs on the bottom depict total surviving cells in the retinal ganglion cell layer. p values are versus the PBS injected group at 48 hr or 7 d (**p<0.01).

FIGS. 7A and 7B are images of graphs illustrating the effect of various PEDF fragments after transient ischemia. FIG. 7A is a graph of the mean thickness of INL and IPL in retinas 7 days after ischemic injury and treatment. FIG. 7B is a graph of the total surviving cells in the retinal ganglion cell layer P values are from one-way ANOVA measurement compared with control peptide PEDF₁₃₆₋₁₅₅ injected group. (*p<0.05 and **p<0.01).

FIG. 8 is an image of a scanning electron micrograph of PLGA nanospheres loaded with PEDF₈₂₋₁₂₁. Nanospheres were reasonably homogeneous with a mean diameter of approximately 300 nm.

FIG. 9 is a graph of the cumulative in vitro release of PEDF₈₂₋₁₂₁ from PLGA nanospheres in PBS at 37° C. The amount of PEDF₈₂₋₁₂₁ released is normalized per mg of nanospheres for all time-points. The results are presented as the means±standard deviation for three separate experiments.

FIGS. 10A-10C are a series of images and graphs of data exhibiting morphologic and morphometric change of inner retina. Animals were subjected to ischemic injury and immediately after, intravitreously injected with PEDF₈₂₋₁₂₁ peptide (peptide), blank nanosphere (blank MP) or PEDF₈₂₋₁₂₁ peptide nanosphere (peptide MP) and allowed to recover for 7 days. FIG. 10A depicts representative images of hematoxylin and eosin-stained of retinas. Calibration, 20 μm. FIG. 10B is a graph of the mean thickness of INL and IPL in retinas 7 days after ischemic injury and treatment. FIG. 10C is a graph of the total surviving cells in the retinal ganglion cell layer One-way ANOVA measurement was used for statistic analysis, *p is versus the blank nanosphere-injected group. #p is versus the PEDF₈₂₋₁₂₁ peptide nanosphere-injected group (*p<0.05, **p<0.01, ***p<0.001, #p<0.05).

FIG. 11 is a graph of total surviving cells in the retinal ganglion cell layer 7 days after ischemic injury and injection of PBS, PLGA nanospheres (PLGA-blank) or PLGA nanospheres loaded with PEDF₈₂₋₁₂₁ (PLGA-PEDF₈₂₋₁₂₁). p is versus PLGA-blank injected group. (***p<0.001).

FIGS. 12A-12C are a series of images of retinas after transient ischemia injury, followed by injection of various compounds. FIG. 12A are images of normal retina. The left image is immunocytochemically stained for GFAP expression. The right image is DAPI stained. FIG. 12B are GFAP-stained images of retinas 48 hours or 7 days after transient ischemia injury, followed by administration of compounds, as indicated. FIG. 12C are images of GFAP-stained retinas 7 days after ischemic injury and injection of either blank PLGA nanospheres (PLGA-blank) or PEDF₈₂₋₁₂₁-loaded PLGA nanospheres (PLGA-PEDF₈₂₋₁₂₁). Bar=20 μm.

FIGS. 13A and 13B are a series of images of a denaturing protein gel and a Western blot of bacterially-expressed, codon-optimized human PEDF fusion protein (coPEDF). FIG. 13A is an image of a Coomassie-stained SDS-PAGE gel with two lanes of purified coPEDF. Molecular weights markers are indicated on the left side. FIG. 13B is an image of a Western blot. PEDF and coPEDF are indicated. Molecular weight markers are shown on the right.

FIG. 14 is an image of a Western blot of increasing amounts of purified coPEDF run on a 10% SDS-PAGe gel. Lane 1: 1 μg coPEDF. Lane 2: 2 μg coPEDF. Lane 3: 5 μg coPEDF. Molecular weight markers are indicated on the left.

### DETAILED DESCRIPTION OF THE INVENTION

The invention provides a peptide that is a fragment of full-length PEDF that retains the activity of the full-length PEDF. The invention further provides compositions comprising the peptide, methods of using such compositions and kits containing a peptide or composition of the invention.

### DEFINITIONS

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and nucleic acid chemistry and hybridization are those well known and commonly employed in the art.

Standard techniques are used for nucleic acid and peptide synthesis. The techniques and procedures are generally performed according to conventional methods in the art and various general references (e.g., Sambrook and Russell, 2001, Molecular Cloning, A Laboratory Approach, Cold Spring Harbor Press, Cold Spring Harbor, N.Y., and Ausubel et al., 2005, Current Protocols in Molecular Biology, John Wiley & Sons, NY), which are provided throughout this document.

The nomenclature used herein and the laboratory procedures used in analytical chemistry and organic syntheses described below are those well known and commonly employed in the art. Standard techniques or modifications thereof, are used for chemical syntheses and chemical analyses.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

It is understood that any and all whole or partial integers between any ranges set forth herein are included herein.

The term “about” will be understood by persons of ordinary skill in the art and will vary to some extent on the context in which it is used.

As used herein, a “disease” is a state of health of an animal wherein the animal cannot maintain homeostasis, and wherein, if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, a disease or disorder is “alleviated” if the severity of a symptom of the disease or disorder, the frequency with which such a symptom is experienced by a patient, or both, are reduced.

As used herein, a “therapeutically effective amount” refers to a nontoxic but sufficient amount of an agent to provide the desired biological result. That result can be reduction and/or alleviation of the signs, symptoms, or causes of a disease or disorder, or any other desired alteration of a biological system. An appropriate therapeutic amount in any individual case may be determined by one of ordinary skill in the art using routine experimentation.

A “therapeutic” treatment is a treatment administered to a subject who exhibits signs of pathology for the purpose of diminishing or eliminating those signs.

A “prophylactic” treatment is a treatment administered to a subject who does not exhibit signs of a disease or exhibits only early signs of the disease for the purpose of decreasing the risk of developing pathology associated with the disease.

A “subject” of treatment is a mammal, including a human. Non-human animals subject to diagnosis or treatment in the methods of the invention include, for example, primates, cattle, goats, sheep, horses, dogs, cats, mice, rats, and the like.

“Polypeptide,” as used herein, refers to a polymer composed of amino acid residues, related naturally-occurring structural variants, and synthetic non-naturally-occurring variants thereof, linked via peptide bonds. Synthetic polypeptides can be synthesized, for example, using an automated polypeptide synthesizer.

The term “protein” typically refers to large polypeptides.

The term “peptide” typically refers to short polypeptides.

Conventional notation is used herein to portray polypeptide sequences: the left-hand end of a polypeptide sequence is the amino-terminus (N-terminus); the right-hand end of a polypeptide sequence is the carboxyl-terminus (C-terminus).

The term “substantially pure” describes a compound, e.g., a protein or polypeptide, which has been separated from components which naturally accompany it. Typically, a compound is substantially pure when at least 10%, more preferably at least 20%, more preferably at least 50%, more preferably at least 60%, more preferably at least 75%, more preferably at least 90%, and most preferably at least 99% of the total material (by volume, by wet or dry weight, or by mole percent or mole fraction) in a sample is the compound of interest. Purity can be measured by any appropriate method, e.g., in the case of polypeptides, by column chromatography, gel electrophoresis or HPLC analysis. A compound, e.g., a protein, is also substantially purified when it is essentially free of naturally associated components or when it is separated from the native contaminants which accompany it in its natural state.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

In the context of the present invention, the following abbreviations for the commonly occurring nucleic acid bases are used. “A” refers to adenosine, “C” refers to cytidine, “G” refers to guanosine, “T” refers to thymidine, and “U” refers to uridine.

A “polynucleotide” means a single strand or parallel and anti-parallel strands of a nucleic acid. Thus, a polynucleotide may be either a single-stranded or a double-stranded nucleic acid.

The term “nucleic acid” typically refers to large polynucleotides.

The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

As used herein, “recombinant polynucleotide” refers to a polynucleotide having sequences that are not naturally joined together. An amplified or assembled recombinant polynucleotide may be included in a suitable vector, and the vector can be used to transform a suitable host cell. A recombinant polynucleotide may serve a non-coding function (e.g., promoter, origin of replication, ribosome-binding site, etc.) as well.

As used herein, a “recombinant polypeptide” is one which is produced upon expression of a recombinant polynucleotide.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

A “vector” is a composition of matter which comprises an isolated nucleic acid and which can be used to deliver the isolated nucleic acid to the interior of a cell.

As used herein, as “expression vector” refers to a vector comprising a recombinant polynucleotide comprising an expression cassette. An expression vector comprises sufficient cis-acting elements for expression; other elements for expression can be supplied by the host cell or in an in vitro expression system. Expression vectors useful in the invention include all those known in the art, such as cosmids, plasmids (e.g., naked or contained in liposomes) and viruses that incorporate the recombinant polynucleotide.

By “expression cassette” is meant a nucleic acid molecule comprising a coding sequence operably linked to promoter/regulatory sequences necessary for transcription and translation of the coding sequence.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

By describing two peptides or polypeptides as “operably fused” is meant that the structure and/or biological activity of each individual peptide is also present in the fusion.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive promoter” is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

A host cell that comprises a recombinant polynucleotide is referred to herein as a “recombinant host cell.” A gene that is expressed in a recombinant host cell, wherein the gene comprises a recombinant polynucleotide, produces a recombinant polypeptide.

By the term “signal sequence” is meant a polynucleotide sequence which encodes a peptide that directs the path a polypeptide takes within a cell, i.e., it directs the cellular processing of a polypeptide in a cell, including, but not limited to, eventual secretion of a polypeptide from a cell. A signal peptide is a sequence of amino acids which are typically, but not exclusively, found at the amino terminus of a polypeptide which targets the synthesis of the polypeptide to the endoplasmic reticulum. In some instances, the signal peptide is proteolytically removed from the polypeptide and is thus absent from the mature protein.

“Homologous,” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC5′ share 50% homology. As used herein, “homology” is used synonymously with “identity.”

As used herein, a “peptidomimetic” is a compound containing non-peptidic structural elements that is capable of mimicking the biological action of a parent peptide. A peptidomimetic may or may not comprise peptide bonds.

“Instructional material,” as that term is used herein, includes a publication, a recording, a diagram, or any other medium of expression which can be used to communicate the usefulness of the composition and/or compound of the invention in a kit. The instructional material of the kit may, for example, be affixed to a container that contains the compound and/or composition of the invention or be shipped together with a container which contains the compound and/or composition. Alternatively, the instructional material may be shipped separately from the container with the intention that the recipient uses the instructional material and the compound cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

As used herein, “PEDF₈₂₋₁₂₁” refers to a peptide consisting of about residues 82 to 121 of a human pigment epithelium derived factor or the homologous region from a naturally-occurring PEDF homolog. “About” as used with respect to PEDF₈₂₋₁₂₁ means±3 flanking residues from a naturally-occurring human PEDF homolog at either or both termini of the peptide.

As used herein, “PEDF₇₆₋₉₅” refers to a peptide consisting of about residues 76 to 95 of a human pigment epithelium derived factor or the homologous region from a naturally-occurring PEDF homolog. “About” as used with respect to PEDF₇₆₋₉₅ means±3 flanking residues of a naturally-occurring human PEDF homolog at either or both termini of the peptide.

As used herein, “PEDF₉₁₋₁₁₀” refers to a peptide consisting of about residues 91 to 110 of a human pigment epithelium derived factor or the homologous region from a naturally-occurring PEDF homolog. “About” as used with respect to PEDF₉₁₋₁₁₀ means±3 flanking residues of a naturally-occurring human PEDF homolog at either or both termini of the peptide.

As used herein, “PEDF₁₀₆₋₁₂₅” refers to a peptide consisting of about residues 106 to 125 of a human pigment epithelium derived factor or the homologous region from a naturally-occurring PEDF homolog. “About” as used with respect to PEDF₁₀₆₋₁₂₅ means±3 flanking residues of a naturally-occurring human PEDF homolog at either or both termini of the peptide.

“Neurotrophic activity” as used herein is the ability to enhance survival and/or growth of neuronal cell populations. Neuroprotective activity, such as delaying or reducing neuronal apoptosis, is encompassed by the term neurotrophic activity.

### DESCRIPTION

The invention springs in part from the discovery that an about 40 residue fragment of pigment epithelium-derived factor (PEDF) retains the neurotrophic activity of full-length PEDF. Specifically, it has been discovered that a fragment of PEDF consisting of residues 82 to 121 of a human PEDF protein mimics the cell survival effect of PEDF. As demonstrated herein, PEDF₈₂₋₁₂₁ is as effective as full-length PEDF in protecting retinal ganglion cells (RGC) from apoptosis in eyes subjected to ischemic injury. Both PEDF and PEDF₈₂₋₁₂₁ delay retinal responses to ischemic injury. It is also shown that PEDF₈₂₋₁₂₁ protects the integrity of other layers of the inner retina, including the inner plexiform layer (IPL), that are important to ganglion cell function. Furthermore, it has been discovered that delivering PEDF₈₂₋₁₂₁ in poly (lactide-co-glycolide) PLGA nanospheres significantly prolongs its protective effect in vivo with no noticeable side effects. Additionally, it has been discovered that a peptide consisting of residues 76 to 95 of PEDF (PEDF₇₆₋₉₅) protects the INL and IPL and prevents them from thinning after ischemic retinal injury. A peptide consisting of residues 91 to 110 of PEDF (PEDF₉₁₋₁₁₀) has been discovered to protect RGCs. Specifically, it has been discovered that PEDF₉₁₋₁₁₀ protects about 85% of RGCs from dying after ischemic injury A peptide consisting of residues 106-125 of PEDF (PEDF₁₀₆₋₁₂₅) also protects RGCs from dying after ischemic injury.

Thus, the invention provides PEDF peptides with neurotrophic activity and a composition, useful for a wide variety of applications, comprising one or more PEDF peptides The invention also provides methods of using the peptides as well as kits containing the peptides. The small peptides of the invention advantageously allow the relative ease of crossing tissue barriers, of providing active doses, of ease of synthesis in reproducibly pure large-scale quantities, and fewer side effects, compared to full length PEDF.

The invention further provides an improved method of expressing full-length human PEDF in bacteria using a codon-optimized coding sequence and fusion protein comprising a full-length, human PEDF having improved properties compared to naturally-occurring human PEDF.

PEDF Peptides and Fusion Proteins

The invention provides several N-terminal PEDF peptides and isolated nucleic acids encoding them. Also provided are vectors and cells comprising an isolated nucleic acid of the invention. The peptides are PEDF₈₂₋₁₂₁, PEDF₇₆₋₉₅, PEDF₉₁₋₁₁₀ and PEDF₁₀₆₋₁₂₅ (where the numbering refers to the human amino acid sequence including the signal peptide sequence), and variants thereof that retain their biological activity. The PEDF peptides of the invention can be from any organism. Preferably, to avoid the possibility of antigenicity of a xeno-PEDF and a resultant anti-xeno-PEDF immune response, the PEDF peptide is the same species as the subject to which it is administered. For instance, a human PEDF₈₂₋₁₂₁ is preferable in the treatment of humans.

PEDF genes have been cloned and sequenced in many mammals, including human, mouse (Mus musculus), cow (Bos taurus), and rat (Rattus norvegicus). Sequence alignment analysis to identify the residues in a non-human PEDF gene that correspond to residues 82-121, 76-95, 91-110 and 106-125 is well known in the art, and the skilled artisan is able to do so without undue experimentation.

In preferred embodiments, the PEDF peptides of the invention are human. The nucleotide and amino acid sequences for a full-length human PEDF are SEQ ID NOS: 1 and 2, respectively. The amino acid sequence of a human PEDF₈₂₋₁₂₁ of the invention is PLSVATALSALSLGAEQRTESIIHRALYYDLISSPDIHGT (SEQ ID NO: 3). An exemplary coding sequence for human PEDF₈₂₋₁₂₁ is provided in SEQ ID NO: 4. Another exemplary coding sequence, useful for expression in bacteria, is nucleotides 244-363 of SEQ ID NO: 11. A naturally-occurring variant of PEDF has DE in place of AE at positions 15 and 16 in SEQ ID NO: 3. The amino acid sequence of a human PEDF₇₆₋₉₅ of the invention is TNVLLSPLSVATALSALSLG (SEQ ID NO: 5), and an exemplary coding sequence is provided in SEQ ID NO: 6. A second exemplary coding sequence, useful for expression in bacteria, is nucleotides 226-285 of SEQ ID NO: 11. The amino acid sequence of a human PEDF₉₁₋₁₁₀ of the invention is ALSLGAEQRTESIIHRALYY (SEQ ID NO: 7). SEQ ID NO: 8 is a coding sequence for SEQ ID NO: 7. The sequence of nucleotides 271-330 of SEQ ID NO: 11 is another exemplary coding sequence for PEDF₉₁₋₁₁₀ of the invention. The amino acid sequence of a human PEDF₁₀₆₋₁₂₅ of the invention is RALYYDLISSPDIHGTYKEL (SEQ ID NO: 9). SEQ ID NO: 10 is an exemplary coding sequence for SEQ ID NO: 9. Another exemplary coding sequence for PEDF₁₀₆₋₁₂₅, useful for expression in bacteria, is nucleotides 316-375 of SEQ ID NO: 11. The skilled artisan comprehends that numerous other coding sequences can encode the same amino acid sequences and designing them is within ordinary skill in the art.

Variants of PEDF₈₂₋₁₂₁, PEDF₇₆₋₉₅ PEDF₉₁₋₁₁₀ and PEDF₁₀₆₋₁₂₅ can also be used in the methods of the invention. In one embodiment, variants differ from naturally-occurring peptides by conservative amino acid sequence differences or by modifications which do not affect sequence, or by both.

For example, conservative amino acid changes may be made, which although they alter the primary sequence of the peptide, do not normally alter its function. Conservative amino acid substitutions typically include substitutions within the following groups:
glycine, alanine;
valine, isoleucine, leucine;
aspartic acid, glutamic acid;
asparagine, glutamine;
serine, threonine;
lysine, arginine;
phenylalanine, tyrosine.

There are several PEDF structure-function analyses that have been performed such that the skilled artisan has sufficient guidance for identifying sites for mutation in PEDF₈₂₋₁₂₁, PEDF₇₆₋₉₅, PEDF₉₁₋₁₁₀ and PEDF₁₀₆₋₁₂₅ that are not likely to eliminate the neurotrophic activity of the peptide. For instance, Simonovic et al. have determined the crystal structure of human PEDF (2001, Proc Natl Acad Sci USA 98:11131-11135). Tombran-Tink et al. (2005, J Struc Biol. 151:130-150) analyzed sequence alignments of PEDF genes from multiple species to identify conserved structural domains. Sequence analysis using conventional methods known in the art of naturally-occurring homologs allows one of skill in the art to recognize sites that tolerate mutation and those that are conserved. Thus, there is sufficient guidance in the art for the skilled artisan to design and prepare variants of PEDF₈₂₋₁₂₁, PEDF₇₆₋₉₅, PEDF₉₁₋₁₁₀ and PEDF₁₀₆₋₁₂₅ for use in the present invention.

Variants of PEDF₈₂₋₁₂₁, PEDF₇₆₋₉₅, PEDF₉₁₋₁₁₀ and PEDF₁₀₆₋₁₂₅ based on sequence identity are also included in the present invention. Preferably, such variants do not have increased immunogenicity compared to the non-variant PEDF peptide parent molecule. Preferably, the PEDF₈₂₋₁₂₁ variant molecule used in the invention has at least about 60% sequence identity to SEQ ID NO: 3, preferably at least about 65%, 70%, 80%, 90% and more preferably still about 95% sequence identity to SEQ ID NO: 3 and retains PEDF₈₂₋₁₂₁ activity. Preferably, the PEDF₇₆₋₉₅ variant molecule used in the invention has at least about 60% sequence identity to SEQ ID NO: 5, preferably at least about 65%, 70%, 80%, 90% and more preferably still about 95% sequence identity to SEQ ID NO: 5 and retains PEDF₇₆₋₉₅ activity. Preferably, the PEDF₉₁₋₁₁₀ variant molecule used in the invention has at least about 60% sequence identity to SEQ ID NO: 7, preferably at least about 65%, 70%, 80%, 90% and more preferably still about 95% sequence identity to SEQ ID NO: 7 and retains PEDF₉₁₋₁₁₀ activity. Preferably, the PEDF₁₀₆₋₁₂₅ variant molecule used in the invention has at least about 60% sequence identity to SEQ ID NO: 9, preferably at least about 65%, 70%, 80%, 90% and more preferably still about 95% sequence identity to SEQ ID NO: 9 and retains PEDF₁₀₆₋₁₂₅ activity. PEDF peptide activity can be measured, for instance, by assessing neutrotrophic activity using methods known in the art. Methods of assessing neurotrophic activity are also disclosed in the Examples herein.

The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site having the universal resource locator http://www(dot)ncbi(dot)nlm(dot)nih(dot)gov/BLAST/. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used. See http://www(dot)ncbi(dot)nlm(dot)nih(dot)gov.

The percent identity between two sequences can be determined using techniques similar to those described above, with or without allowing gaps. In calculating percent identity, exact matches are typically counted.

PEDF peptides of the invention may also have modifications. Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are PEDF peptides which have been modified using ordinary molecular biological techniques so as to improve their resistance to proteolytic degradation or to optimize solubility properties or to render them more suitable as a therapeutic agent. Such variants include those containing residues other than naturally-occurring L-amino acids, e.g., D-amino acids or non-naturally-occurring synthetic amino acids. The PEDF peptides of the invention may further be conjugated to non-amino acid moieties that are useful in their therapeutic application. In particular, moieties that improve the stability, biological half-life, water solubility, and/or immunologic characteristics of the peptide are useful. A non-limiting example of such a moiety is polyethylene glycol (PEG).

Covalent attachment of biologically active compounds to water-soluble polymers is one method for alteration and control of biodistribution, pharmacokinetics, and often, toxicity for these compounds (Duncan et al., 1984, Adv. Polym. Sci. 57:53-101). Many water-soluble polymers have been used to achieve these effects, such as poly(sialic acid), dextran, poly(N-(2-hydroxypropyl)methacrylamide) (PHPMA), poly(N-vinylpyrrolidone) (PVP), poly(vinyl alcohol) (PVA), poly(ethylene glycol-co-propylene glycol), poly(N-acryloyl morpholine (PAcM), and poly(ethylene glycol) (PEG) (Powell, 1980, Polyethylene glycol. In R. L. Davidson (Ed.) Handbook of Water Soluble Gums and Resins. McGraw-Hill, New York, chapter 18). PEG possess an ideal set of properties: very low toxicity (Pang, 1993, J. Am. Coll. Toxicol. 12: 429-456) excellent solubility in aqueous solution (Powell, supra), low immunogenicity and antigenicity (Dreborg et al., 1990, Crit. Rev. Ther. Drug Carrier Syst. 6: 315-365). PEG-conjugated or “PEGylated” protein therapeutics, containing single or multiple chains of polyethylene glycol on the protein, have been described in the scientific literature (Clark et al., 1996, J. Biol. Chem. 271: 21969-21977; Hershfield, 1997, Biochemistry and immunology of poly(ethylene glycol)-modified adenosine deaminase (PEG-ADA). In J. M. Harris and S. Zalipsky (Eds) Poly(ethylene glycol): Chemistry and Biological Applications. American Chemical Society, Washington, D.C., p 145-154; Olson et al., 1997, Preparation and characterization of poly(ethylene glycol)ylated human growth hormone antagonist. In J. M. Harris and S. Zalipsky (Eds) Poly(ethylene glycol): Chemistry and Biological Applications. American Chemical Society, Washington, D.C., p 170-181).

Also included in the invention are fusion proteins comprising a PEDF₈₂₋₁₂₁ PEDF₇₆₋₉₅, PEDF₉₁₋₁₁₀ or PEDF₁₀₆₋₁₂₅ peptide portion. The PEDF peptide portion consists of any of the previously described PEDF peptide molecules (PEDF₈₂₋₁₂₁, PEDF₇₆₋₉₅, PEDF₉₁₋₁₁₀ or PEDF₁₀₆₋₁₂₅), including naturally-occurring homologs, variants and modified forms thereof. Hybrid polypeptides may comprise peptide sequences at the N-terminal of a PEDF peptide portion, at the C-terminal of a PEDF peptide portion or both. These additional segments may be any amino acid sequence useful for the purification, identification and/or therapeutic or prophylactic application of the peptide. Non-limiting examples of such additional segments include LacZ, FLAG-tag, Myc, His₆ and the like. A cellular targeting sequence can be used to increase local concentration of PEDF peptide at a desired cell type, for instance, a retinal ganglion cell. Fusion proteins of the invention may be modified as described elsewhere herein, for instance, to improve stability, biological stability or pharmokinetics. The PEDF peptide may be fused directly to the second peptide or may be separated by a linker sequence. Linker sequences are well known in the art.

In one embodiment, a fusion protein comprises a first peptide operably fused to a second peptide. The first peptide consists essentially of a sequence selected from the group consisting of residues 82 to 121 of a human pigment epithelium-derived factor (PEDF), residues 76 to 95 of a human PEDF, residues 91 to 110 of a human PED, residues 106 to 125 of a human PEDF F and variants thereof. The second peptide is not a PEDF peptide.

In another embodiment, a fusion protein comprises a first peptide operably fused to a second peptide. The first peptide consists essentially of a sequence selected from the group consisting of residues 82 to 121 of a human pigment epithelium-derived factor (PEDF), residues 76 to 95 of a human PEDF, residues 91 to 110 of a human PED, residues 106 to 125 of a human PEDF F and variants thereof. The second peptide is a PEDF peptide that consists essentially of a sequence selected from the group consisting of residues 82 to 121 of a human pigment epithelium-derived factor (PEDF), residues 76 to 95 of a human PEDF, residues 91 to 110 of a human PEDF, residues 106 to 125 of a human PEDF, a PEDF fragment having anti-angiogenic activity, such as residues 44 to 77 of a human PEDF, a C-terminal fragment of a human PEDF comprising a sequence unique to PEDF as described in Tombran-Tink et al. (2005, J Struc Biol. 151:130-150) and variants thereof, wherein said first and second peptides are not identical to each other.

The invention includes an isolated nucleic acid encoding a fusion protein, a vector comprising the isolated nucleic acid and a cell comprising the isolated nucleic acid.

Peptidomimetics of PEDF₈₂₋₁₂₁, PEDF₇₆₋₉₅, PEDF₉₁₋₁₁₀ and PEDF₁₀₆₋₁₂₅ are also useful in the method of the invention and are encompassed by the invention.

Nucleic acid sequences encoding variants of PEDF₈₂₋₁₂₁, PEDF₇₆₋₉₅, PEDF₉₁₋₁₁₀ and PEDF₁₀₆₋₁₂₅ variants are also included in the invention. For instance, a sequence that hybridizes to SEQ ID NO: 4 under stringent hybridization conditions and encodes a polypeptide with PEDF₈₂₋₁₂₁ activity can be used in the methods of the invention.

As used herein, “stringent hybridization conditions” refers to conditions for hybridization and washing under which nucleotide sequences typically remain hybridized to each other. Such stringent conditions are known to those skilled in the art and may be found, for instance, in Ausubel et al. (Current Protocols in Molecular Biology, John Wiley & Sons, New York (2005)). A preferred example of stringent hybridization conditions is hybridization in 6× sodium chloride/sodium citrate (SSC) at about 45° C., followed by one or more washes in 0.2×SSC, 0.1% SDS at 50° C. Washes may be performed at higher temperatures, such as, but not limited to 55° C., 60° C. and 65° C. Preferably, an isolated nucleotide that hybridizes under stringent conditions to SEQ ID NO. 4 corresponds to a naturally-occurring nucleotide. As used herein, a “naturally-occurring nucleotide” refers to an RNA or DNA molecule having a nucleotide sequence that occurs in nature (i.e. encodes a natural protein).

The PEDF peptides of the invention may be prepared by chemical or biological means. Biological methods include, without limitation, expression of a nucleic acid encoding an PEDF peptide in a host cell or in an in vitro translation systems.

The polypeptides may be chemically synthesized by Merrifield-type solid phase peptide synthesis. This method may be routinely performed to yield peptides up to about 60-70 residues in length, and may, in some cases, be utilized to make peptides up to about 100 amino acids long. Larger peptides may also be generated synthetically via fragment condensation or native chemical ligation (Dawson et al., 2000, Ann. Rev. Biochem. 69:923-960). An advantage to the utilization of a synthetic peptide route is the ability to produce large amounts of peptides, even those that rarely occur naturally, with relatively high purities, i.e., purities sufficient for research, diagnostic or therapeutic purposes.

Solid phase peptide synthesis is described by Stewart et al. in Solid Phase Peptide Synthesis, 2nd Edition, 1984, Pierce Chemical Company, Rockford, Ill.; and Bodanszky and Bodanszky in The Practice of Peptide Synthesis, 1984, Springer-Verlag, New York. At the outset, a suitably protected amino acid residue is attached through its carboxyl group to a derivatized, insoluble polymeric support, such as cross-linked polystyrene or polyamide resin. “Suitably protected” refers to the presence of protecting groups on both the α-amino group of the amino acid, and on any side chain functional groups. Side chain protecting groups are generally stable to the solvents, reagents and reaction conditions used throughout the synthesis, and are removable under conditions which will not affect the final peptide product. Stepwise synthesis of the oligopeptide is carried out by the removal of the N-protecting group from the initial amino acid, and coupling thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group, such as formation into a carbodiimide, a symmetric acid anhydride, or an “active ester” group, such as hydroxybenzotriazole or pentafluorophenyl esters.

Examples of solid phase peptide synthesis methods include the BOC method which utilized tert-butyloxcarbonyl as the α-amino protecting group, and the FMOC method which utilizes 9-fluorenylmethyloxcarbonyl to protect the α-amino of the amino acid residues, both which methods are well-known by those of skill in the art.

Incorporation of N- and/or C-blocking groups may also be achieved using protocols conventional to solid phase peptide synthesis methods. For incorporation of C-terminal blocking groups, for example, synthesis of the desired peptide is typically performed using, as solid phase, a supporting resin that has been chemically modified so that cleavage from the resin results in a peptide having the desired C-terminal blocking group. To provide peptides in which the C-terminus bears a primary amino blocking group, for instance, synthesis is performed using a p-methylbenzhydrylamine (MBHA) resin, so that, when peptide synthesis is completed, treatment with hydrofluoric acid releases the desired C-terminally amidated peptide. Similarly, incorporation of an N-methylamine blocking group at the C-terminus is achieved using N-methylaminoethyl-derivatized DVB, resin, which upon HF treatment releases a peptide bearing an N-methylamidated C-terminus. Blockage of the C-terminus by esterification can also be achieved using conventional procedures. This entails use of resin/blocking group combination that permits release of side-chain peptide from the resin, to allow for subsequent reaction with the desired alcohol, to form the ester function. FMOC protecting group, in combination with DVB resin derivatized with methoxyalkoxybenzyl alcohol or equivalent linker, can be used for this purpose, with cleavage from the support being effected by TFA in dicholoromethane. Esterification of the suitably activated carboxyl function, e.g. with DCC, can then proceed by addition of the desired alcohol, followed by de-protection and isolation of the esterified peptide product.

Incorporation of N-terminal blocking groups may be achieved while the synthesized peptide is still attached to the resin, for instance by treatment with a suitable anhydride and nitrile. To incorporate an acetyl blocking group at the N-terminus, for instance, the resin-coupled peptide can be treated with 20% acetic anhydride in acetonitrile. The N-blocked peptide product may then be cleaved from the resin, de-protected and subsequently isolated.

Biological preparation of a peptide of the invention involves expression of a nucleic acid encoding a PEDF peptide. An expression cassette comprising such a coding sequence may be used to produce a PEDF peptide for use in the method of the invention.

In the context of an expression vector, the vector can be readily introduced into a host cell, e.g., mammalian, bacterial, yeast or insect cell by any method in the art. Coding sequences for a PEDF peptide of the invention may be codon optimized based on the codon usage of the intended host cell in order to improve expression efficiency as demonstrated herein. Codon usage patterns can be found in the literature (Nakamura et al., 2000, Nuc Acids Res. 28:292). Representative examples of appropriate hosts include bacterial cells, such as streptococci, staphylococci, E. coli, Streptomyces and Bacillus subtilis cells; fungal cells, such as yeast cells and Aspergillus cells; insect cells such as Drosophila S2 and Spodoptera Sf9 cells; animal cells such as CHO, COS, HeLa, C127, 3T3, BHK, HEK 293 and Bowes melanoma cells; and plant cells.

The expression vector can be transferred into a host cell by physical, biological or chemical means. Physical methods for introducing a polynucleotide into a host cell include calcium phosphate precipitation, lipofection, particle bombardment, microinjection, electroporation, photoporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York), and in Ausubel et al. (2005, Current Protocols in Molecular Biology, John Wiley & Sons, New York). Thus, the invention encompasses expression vectors encoding a PEDF peptide or fusion protein of the invention, as well as cells comprising such vectors.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from baculovirus, papovavirus, vaccinia virus, pseudorabies virus, fowl pox virus, lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. A preferred colloidal system for use as a delivery vehicle in vitro and in vivo is a liposome (i.e., an artificial membrane vesicle). The preparation and use of such systems is well known in the art.

In the case where a non-viral delivery system is utilized, a preferred delivery vehicle is a liposome. The above-mentioned delivery systems and protocols can be found in Gene Targeting Protocols, 2^{nd} ed., Kmiec ed., Humana Press, Totowa, N.J., pp 1-35 (2002) and Gene Transfer and Expression Protocols, Vol. 7, (Methods in Molecular Biology) Murray ed., Humana Press, Totowa, N.J., pp 81-89 (1991).

Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

To ensure that the polypeptide obtained from either chemical or biological synthetic techniques is the desired polypeptide, analysis of the peptide composition can be conducted. Such amino acid composition analysis may be conducted using high resolution mass spectrometry to determine the molecular weight of the peptide. Alternatively, or additionally, the amino acid content of the peptide can be confirmed by hydrolyzing the peptide in aqueous acid, and separating, identifying and quantifying the components of the mixture using HPLC, or an amino acid analyzer. Protein sequenators, which sequentially degrade the peptide and identify the amino acids in order, may also be used to determine definitely the sequence of the peptide.

Prior to its use in a method of the invention, the PEDF peptide is purified to remove contaminants. In this regard, it will be appreciated that the peptide will be purified so as to meet the standards set out by the appropriate regulatory agencies. Any one of a number of a conventional purification procedures may be used to attain the required level of purity including, for example, reversed-phase high-pressure liquid chromatography (HPLC) using an alkylated silica column such as C₄-, C₈- or C₁₈-silica. A gradient mobile phase of increasing organic content is generally used to achieve purification, for example, acetonitrile in an aqueous buffer, usually containing a small amount of trifluoroacetic acid. Ion-exchange chromatography can be also used to separate polypeptides based on their charge. Affinity chromatography is also useful in purification procedures.

In preparing a substantially pure polypeptide, an immunological, enzymatic or other assay can be used to monitor purification at each stage in the procedure. Protein purification methods are well known in the art, and are described, for example in Deutscher et al. (ed., 1990, Guide to Protein Purification, Harcourt Brace Jovanovich, San Diego, Calif.).

Uses for the Inventive Peptides

The PEDF peptides disclosed herein are useful in any therapeutic or prophylactic treatment known in the art, or subsequently discovered, for full-length PEDF protein. Such therapeutic applications are disclosed in detail in, for instance, U.S. Pat. Nos. 6,451,763; 6,288,024; 6,391,850; 6,670,333; 6,797,691; 6,919,309 and US Patent Publication 20060008900, each of which is enclosed here in its entirety. In preferred embodiments, the disease is one involving neuronal degeneration which would therefore benefit from the neurotrophic activity of the peptides disclosed herein. Methods of treatment may be therapeutic or prophylactic.

Diseases or disorders involving neuronal degeneration, include, but are not limited to, nerve injuries, neurodegenerative diseases, and ocular diseases and disorders, including, but not limited to, transient or chronic ischemic injury. In particular, nerve injuries and neurodegenerative diseases of neurons in the retina, brain and spinal cord are beneficially treated using a PEDF peptide of the invention. Non-limiting examples of such diseases and disorders include Parkinson's disease, Huntington's disease, Alzheimer's disease, amyotrophic lateral sclerosis, multiple sclerosis, diabetic retinopathy, retinopathy of prematurity, macular degeneration, glaucoma, venous stasis retinopathy, ischemic oculopathy and ocular ischemic syndrome.

Neural transplantation can be used to treat nerve injuries and neurodegenerative diseases. Treatment of neuronal cells with compositions of the invention to enhance nerve cell survival is also embraced by the invention. Treatment may occur before, during or after neural transplantation. Similarly, transfection of either neurons or astroglia with an expression vector comprising a coding sequence for a PEDF peptide of the invention before implantation can provide a long-term source of a PEDF peptide at the transplantation site. Transplantation of neural retina and photoreceptor cells is contemplated to benefit from the treatment with a composition of the invention of the invention before, during or after transplantation. Alternatively, an expression vector comprising a coding sequence for a PEDF peptide of the invention can be transfected at high levels into adjacent retinal pigment epithelial (RPE) cells where they can serve as a source of a PEDF peptide.

The peptides of the invention are also useful in prolonging neuronal cell survival in in vitro cultures.

Modified PEDF and Bacterial Expression

A codon-optimized coding sequence for PEDF is provided in SEQ ID NO; 11. The encoded human PEDF sequence is shown in SEQ ID NO: 12. Approximately 25% of the naturally-occurring PEDF nucleotide sequence is changed to codon-optimize for bacterial expression. This coding sequence markedly improves bacterial expression of the encoded human PEDF. In addition, in one embodiment, thioredoxin, a 109 amino acid protein which catalyzes disulfide bond formation, and a His₆ tag are fused N-terminally to the PEDF sequence, yielding a fusion PEDF protein (coPEDF). The PEDF sequence optionally does not include the signal peptide sequence (encoding about the first 19 amino acids of SEQ ID NO: 12).

The invention includes sequence variants of SEQ ID NO: 11. Preferably, the variant molecule has at least about 60% sequence identity to SEQ ID NO: 11, preferably at least about 65%, 70%, 80%, 90% and more preferably still about 95% sequence identity to SEQ ID NO: 11 and encodes a PEDF protein having at least one biological activity selected from neurotrophic activity and anti-angiogenic activity.

The invention includes a vector comprising SEQ ID NO: 11 or variants thereof, as well as a cell comprising such a vector.

Advantageously, the codon-optimized PEDF sequence of the invention is robustly expressed in bacteria compared to a non-codon-optimize sequence for human PEDF. As demonstrated in the Examples, the codon-optimized PEDF sequence is expressed at an approximately 10× higher level compared to expression of a non-codon-optimized sequence for human PEDF in prior art methods. Expression of the codon-optimized sequence human PEDF does not impede or prevent bacterial growth, which occurs with recombinant expression of a naturally-occurring human PEDF in bacteria. Furthermore, unlike naturally-occurring PEDF, coPEDF of the invention is not significantly degraded by endogenous bacterial proteases. Consequently, a considerably higher yield of full-length, non-truncated protein is obtained.

Thus, the invention provides a method for preparing human PEDF in a bacterial expression system. The method comprises introducing an expression cassette comprising SEQ ID NO: 11 into a bacterium and expressing the expression cassette to produce a human PEDF protein. In one embodiment, the expression cassette is in an expression vector, as described elsewhere herein. Optionally, SEQ ID NO: 11 is fused to a sequence encoding thioredoxin and/or a sequence encoding His₆ to produce a fusion protein.

The coPEDF protein unexpectedly has improved efficacy compared to a naturally-occurring human PEDF. Specifically, the modified PEDF has increased VEGFR1 binding compared to the VEGFR1 binding affinity of PEDF purified from human plasma or recombinantly-produced non-modified human PEDF. Moreover, coPEDF blocks VEGFR2 phosphorylation and VEGF mitogenic sigaling with greater effect than recombinant, non-modified human PEDF. Additionally, ELISA assays demonstrate that coPEDF improves binding to VEGF receptor KDR (VEGFR2).

The invention further encompasses variants of coPEDF protein. In one embodiment, variants differ from naturally-occurring peptides by conservative amino acid sequence differences or by modifications which do not affect sequence, or by both, as described elsewhere herein. In addition, variants based on sequence identity, as well as modifications, are also part of the present invention.

The coPEDF proteins of the invention possess the angiogenic and neurotrophic properties of naturally-occurring PEDF. Accordingly, it can be used in any therapeutic or prophylactic method envisioned for PEDF, as described elsewhere herein. Non-limiting applications include treatment of diseases and disorders which comprising angiogenesis as part of their pathology. In particular, diseases and disorders involving pathological VEGFR1 activity, pathological VEGFR2 phosphorylation and/or pathological VEGF mitogenic sigaling are suitable for treatment using coPEDF. coPEDF is also suitable for treatment of neurodegenerative diseases and disorders, nerve injuries and ocular disease and disorders discussed elsewhere herein.

Administration, Dosing and Dosing Schedule

Administration of a PEDF peptide, a fusion protein comprising one or more PEDF peptides, or coPEDF of the invention in a method of treatment can be achieved in a number of different ways, using methods known in the art. Such methods include, but are not limited to, providing exogenous coPEDF, PEDF₈₂₋₁₂₁, PEDF₇₆₋₉₅, PEDF₉₁₋₁₁₀ or PEDF₁₀₆₋₁₂₅ to a subject or expressing a recombinant coPEDF, PEDF₈₂₋₁₂₁, PEDF₇₆₋₉₅, PEDF₉₁₋₁₁₀ or PEDF₁₀₆₋₁₂₅ expression cassette.

In a preferred embodiment, exogenous coPEDF, PEDF₈₂₋₁₂₁, PEDF₇₆₋₉₅, PEDF₉₁₋₁₁₀ or PEDF₁₀₆₋₁₂₅ is administered to a subject. The exogenous coPEDF, PEDF₈₂₋₁₂₁, PEDF₇₆₋₉₅, PEDF₉₁₋₁₁₀ or PEDF₁₀₆₋₁₂₅ peptide may be identical in amino acid sequence to the corresponding residues of the endogenous PEDF protein or may be a different sequence. The exogenous peptide may also be a hybrid or fusion protein to facilitate, for instance, delivery to target cells or efficacy. In one embodiment, a hybrid PEDF₈₂₋₁₂₁ protein may comprise a neuron-specific targeting sequence.

In another embodiment, an expression vector comprising an expression cassette encoding coPEDF, PEDF₈₂₋₁₂₁, PEDF₇₆₋₉₅, PEDF₉₁₋₁₁₀ or PEDF₁₀₆₋₁₂₅ is administered to a subject. An expression cassette may comprise a constitutive or inducible promoter.

Such promoters are well known in the art, as are means for genetic modification. Expression vectors and methods for the introduction of exogenous DNA into cells with concomitant expression of the exogenous DNA in the cells are described, for example, in Sambrook et al. (2001, Molecular Cloning: A Laboratory Manual, Cold Spring Harbor Laboratory Press, Cold Spring Harbor, N.Y.), and in Ausubel et al. (eds, 2005, Current Protocols in Molecular Biology, John Wiley & Sons, New York, N.Y.). In one embodiment, a cell comprising an expression vector of the invention is administered to a subject. Thus, the invention encompasses a cell comprising an isolated nucleic acid encoding a PEDF peptide, fusion protein or coPEDF of the invention.

Any expression vector compatible with the expression of coPEDF, PEDF₈₂₋₁₂₁, PEDF₇₆₋₉₅, PEDF₉₁₋₁₁₀ or PEDF₁₀₆₋₁₂₅ is suitable for use in the instant invention, and can be selected from the group consisting of a plasmid DNA, a viral vector, and a mammalian vector. The expression vector, or a vector that is co-introduced with the expression vector, can further comprise a marker gene. Marker genes are useful, for instance, to monitor transfection efficiencies. Marker genes include: genes for selectable markers, including but not limited to, G418, hygromycin, and methotrexate, and genes for detectable markers, including, but not limited to, luciferase and GFP. The expression vector can further comprise an integration signal sequence which facilitates integration of the isolated polynucleotide into the genome of a target cell.

The therapeutic and prophylactic methods of the invention thus encompass the use of pharmaceutical compositions comprising a PEDF peptide, a fusion protein comprising a PEDF peptide, coPEDF and/or an isolated nucleic acid encoding a PEDF peptide, a fusion protein comprising a PEDF peptide or coPEDF to practice the methods of the invention. The pharmaceutical compositions useful for practicing the invention may be administered to deliver a dose of between 1 ng/kg/day and 100 mg/kg/day. In one embodiment, the invention envisions administration of a dose which results in a concentration of the compound of the present invention between 1 μM and 10 μM in a mammal.

Typically, dosages which may be administered in a method of the invention to an animal, preferably a human, range in amount from 0.5 μg to about 50 mg per kilogram of body weight of the animal. While the precise dosage administered will vary depending upon any number of factors, including but not limited to, the type of animal and type of disease state being treated, the age of the animal and the route of administration. Preferably, the dosage of the compound will vary from about 1 μg to about 10 mg per kilogram of body weight of the animal. More preferably, the dosage will vary from about 3 μg to about 1 mg per kilogram of body weight of the animal.

The compound may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc. The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions that are useful in the methods of the invention may be prepared, packaged, or sold in formulations suitable for ophthalmic, oral, rectal, vaginal, parenteral, topical, pulmonary, intranasal, buccal, or another route of administration. Other contemplated formulations include projected nanoparticles, liposomal preparations, resealed erythrocytes containing the active ingredient, and immunologically-based formulations.

In one embodiment, a composition of the invention is formulated as a nanosphere, microsphere or nanotube. In one embodiment, a PEDF peptide, PEDF peptide fusion protein or coPEDF of the invention is encapsulated in a biocompatible delivery polymer. Preferably the polymer is biodegradable and does not induce inflammation in the subject to which is administered. Non-limiting examples of such polymers include poly-lactic acid, poly-glycolic acid, and copolymers of lactic and glycolic acids (PLGA), a hydrogel, a polymer comprising gelatin, a polymer comprising collagen, and a polymer comprising alginate. Preferably, a PEDF peptide is encapsulated in PLGA. Advantageously, this formulation has been shown herein to prolong the efficacy of the PEDF peptide in vivo with no or minimal side effects. In another embodiment, a PEDF peptide, PEDF peptide fusion protein or coPEDF of the invention is incorporated into an ocular implant for delivery in a controlled and sustained manner in the eye. Such ocular implants are known in the art. See, for instance, U.S. Pat. No. 6,713,081, incorporated herein by reference in its entirety.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents.

Controlled- or sustained-release formulations of a pharmaceutical composition of the invention may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intraocular, intravitreal, subcutaneous, intraperitoneal, intramuscular, intrasternal injection, intratumoral, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi-liquid preparations such as liniments, lotions, oil-in-water or water-in-oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers. Such a formulation is administered in the manner in which snuff is taken i.e. by rapid inhalation through the nasal passage from a container of the powder held close to the nares.

Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

A pharmaceutical composition of the invention may be prepared, packaged, or sold in a formulation suitable for ophthalmic administration. Such formulations may, for example, be in the form of eye drops including, for example, a 0.1-1.0% (w/w) solution or suspension of the active ingredient in an aqueous or oily liquid carrier. Such drops may further comprise buffering agents, salts, or one or more other of the additional ingredients described herein. Other ophthalmically-administrable formulations which are useful include those which comprise the active ingredient in microcrystalline form or in a liposomal preparation.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Remington's Pharmaceutical Sciences (1985, Genaro, ed., Mack Publishing Co., Easton, Pa.), which is incorporated herein by reference.

Kits

The invention also includes a kit comprising a PEDF peptide (PEDF₈₂₋₁₂₁, PEDF₇₆₋₉₅, PEDF₉₁₋₁₁₀, PEDF₁₀₆₋₁₂₅ or variants thereof) of the invention and an instructional material which describes, for instance, administering the PEDF peptide to a subject as a prophylactic or therapeutic treatment or a non-treatment use as described elsewhere herein. In an embodiment, this kit further comprises a (preferably sterile) pharmaceutically acceptable carrier suitable for dissolving or suspending the peptide of the invention, for instance, prior to administering the molecule to the animal. Optionally, the kit comprises an applicator for administering the peptide. In one embodiment, the kit comprises a PEDF₈₂₋₁₂₁ peptide. Preferably, the PEDF₈₂₋₁₂₁ peptide is a human PEDF₈₂₋₁₂₁. In one embodiment, PEDF₈₂₋₁₂₁ has the sequence shown in SEQ ID NO: 3, the sequence of residues 82 to 121 of SEQ ID NO: 12 or a sequence variant thereof having neurotrophic activity.

A kit providing a nucleic acid encoding a peptide of the invention and an instructional material is also provided. In one embodiment, the nucleic acid comprises at least one of SEQ ID NO: 4, SEQ ID NO: 6, SEQ ID NO: 8 and SEQ IS NO: 10.

In some embodiments, kits further comprise PLGA suitable for preparing nanospheres comprising a PEDF peptide of the invention.

The invention further provided a kit comprising a coPEDF protein of the invention and an instructional material is provided. In an embodiment, this kit further comprises a (preferably sterile) pharmaceutically acceptable carrier suitable for dissolving or suspending tcoPEDF protein of the invention prior to administering it to a subject. Optionally, the kit comprises an applicator for administering the protein.

A kit providing a nucleic acid comprising a codon-optimized coding sequence for a human PEDF and an instructional material is also provided. In one embodiment, the nucleic acid comprises SEQ ID NO: 11 or a variant thereof. In another embodiment, the nucleic acid comprises about nucleotides 58-1257 of SEQ ID NO: 11 or a variant thereof. Optionally, the nucleic acid further encodes thioredoxin and/or His₆ fused to the codon-optimized sequence to produce a fusion PEDF protein.

The instructional material in each kit simply embody the disclosure provided herein.

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

The materials and methods used in the experiments presented in the Examples are now described.

Preparation of PEDF peptides: PEDF peptides were synthesized chemically using conventional peptide chemistry. Fluoresceinated PEDF₈₂₋₁₂₁ peptide was purchased from SynPep Corp, Dublin, Calif.

Preparation of PEDF peptide PLGA nanospheres: Biodegradable poly (lactide-co-glycolide) (PLGA) nanospheres loaded with a fluoresceinated PEDF₈₂₋₁₂₁ peptide were made using a modification of the double emulsion (water/oil/water) solvent evaporation technique (Luo et al., 1999, Pharm Res. 16(8):1300-1308). Briefly, 200 mg PLGA polymer (Birmingham Polymers, Pelham, Ala.) was fully dissolved in 2 ml dichloromethane in a glass tube. The solution was kept on crushed ice, and 100 μl PEDF peptide (32 μg/μl) was added into the polymer solution while vortexing. The emulsion was sonicated for 10 seconds, 4 ml of 1% PVA stabilizer was added into the tube, and the mixture resonicated until it appeared homogeneous. One hundred (100) ml of 0.3% PVA stabilizer was stirred vigorously in a beaker on a magnetic stir plate. The entire contents of the tube containing polymer and peptide was added to the beaker and stirred for 3 hours to evaporate the dichloromethane. The beaker contents were transferred to four Oak Ridge centrifuge tubes, which were centrifuged at 10,000 g for 5 minutes under 4° C. The supernatant was removed and the solid particles were washed with distilled water. The wash and centrifuge step was repeated 3 times. Nanospheres were resuspended in 3-4 ml distilled water and lyophilized overnight to remove remaining water. The powder was kept at −80° C. until use. The size and homogeneity of the nanospheres was estimated by scanning electron microscopy.

Cumulative release of PEDF peptide from PLGA nanospheres in vitro: Ten (10) mg nanospheres loaded with fluoresceinated PEDF peptide (FITC-peptide) were dissolved in 1.5 ml PBS (pH 7.4) in microcentrifuge tubes and placed in an orbital shaking incubator (37° C.). The tubes were centrifuged at each pre-determined time point; 500 μl of supernatant was removed stored at −80° C. for further analysis. Five hundred (500) μl of fresh PBS was added to the tubes to replace the supernatant withdrawn. The collected supernatants contained the released FITC-peptide. Peptide concentration was determined using a fluorimeter in 96-well format using known unencapsulated FITC-peptide to generate a standard curve. Triplicate data were used for analysis.

Induction of transient retinal ischemia in C57/B16 mice: Animal experiments were conducted in accordance with ARVO guidelines and were approved by the Animal Care and Use Committee of Yale University School of Medicine. Transient retinal ischemia was induced in one eye and the contralateral eye served as control. During the operation and recovery period, animals were placed on water heating pads at 37° C. Eighty-one 10-14 weeks male C57/B16 mice (Charles River Laboratories, Wilmington, Mass., weighing 25-358) were used in this experiment. To induce retinal ischemia, a previously established technique with some modifications was used (Hughes, 1991, Exp Eye Res. 53(5):573-582; Buchi et al., 1991, Opthalmologica 203 (3): 138-147). Briefly, animals were anesthetized by i.p. injection of a mixture of 100 mg/kg Ketamine+10 mg/kg Xylazine. The corneas were topically anesthetized with 0.5% proparacaine hydrochloride (Bausch & Lomb Pharmaceuticals, Inc., Tampa, Fla.), and the pupils dilated with 2.5% phenylephrine (Bausch & Lomb Pharmaceuticals, Inc., Tampa, Fla.). The anterior chambers were cannulated in fully anesthetized animals with a 30-gauge infusion needle connected by PE10 polyethylene tubing and IV set to a physiological saline reservoir. The intraocular pressure (IOP) of the cannulated eye was raised to 120 mmHg for 60 minutes by elevating the saline reservoir. Retinal ischemia was confirmed by direct microscopic observation of whitening of the fundus. The reperfusion was confirmed visually when needle was withdrawn from the anterior chamber. One group of animals was treated with Vetropolycin® ophthalmic ointment (Pharmaderm, Melville, N. Y.) on the ischemic eyes and allowed to recover for 24 hours, 48 hours, 7 days and 14 days before sacrifice. Other groups were kept for intravitreous injections of the PEDF and PEDF peptides.

Intravitreous injection of PEDF, PEDF peptides and encapsulated PEDF peptide: One group of animals remained uninjected to monitor ischemic damage. All other groups received intravitreal injections, which were performed under an ophthalmic microscope. Experimental groups received 1 μl native PEDF protein (1 μg/μl), PEDF₈₂₋₁₂₁ peptide (1 μg/μl), PEDF₇₆₋₉₅ peptide (1 μg/μl), PEDF₉₁₋₁₁₀ peptide (1 μg/μl), PEDF₁₀₆₋₁₂₅ peptide (1 μg/μl) or PLGA PEDF₈₂₋₁₂₁ nanospheres (0.1 mg/μl, less than 1.6 μg PEDF₈₂₋₁₂₁ peptide when fully released) immediately after the end of the transient ischemia. Intravitreous injection was performed with 30-gauge beveled needle attached to PE10 polyethylene tubing and a 10 μl syringe (Hamilton, Reno, Nev.). The injections were performed through the sclera, approximately 1 mm behind the limbus. The needle remained in the vitreous cavity for 30 seconds after injection then gently withdrawn. Control groups were injected with 1 μl PBS, 1 μl control peptide (1 μg/μl), or blank nanospheres (0.1 mg/μl). As a control peptide, a fragment of PEDF, PEDF₁₃₆₋₁₅₅, that has no known neuroprotective function was used. Vetropolycin® ointment was applied to the injected eyes and animals were allowed to recover for 48 hours or 7 days before sacrifice.

Preparation of retinal tissue: at designated time points, mice were sacrificed by carbon dioxide inhalation followed by, in some cases, cervical dislocation. The eyes were enucleated, fixed in 4% paraformaldehyde for 24 hours, washed with PBS and embedded in paraffin. Five (5) μm thick sections that included the optic nerve were collected. Sections were processed for TUNEL, immunocytochemistry, or histochemical staining with hematoxylin and eosin (Surgipath, Ltd, Richmond, Ill.) using standard methods.

Immunocytochemical labeling: Sections were de-paraffined with xylene and re-hydrated. Sections were washed in PBS and antigens were retrieved by placing the slices in boiling 0.1 M sodium citrate solution for 30 minutes followed by washing three times in PBS. After incubation with 5% goat serum at room temperature for 1 hour to block nonspecific binding sites, the sections were treated with a rabbit polyclonal antibody against glial fibriallary acidic protein (GFAP) (Sigma, St. Louis, Mo.) overnight at 4° C. degree in a humid chamber. After washing with PBS, the sections were incubated with fluorescein-labeled anti-rabbit IgG for 1 hour at room temperature. The specimens were then washed and mounted with Vectashield® containing DAPI (Vector Laboratories, Burlingame, Calif.).

Morphologic and Morphometric Analysis: For each eye, three sections, which included the optic disk and were spaced at least 20 μm apart, were used for morphological analysis. The fields analyzed were 4×250 μm zones sampled from all retinal eccentricities. These zones were 500 μm away from the edge of optic disk or the ora serrata. The average measurement of these 4 zones was used for statistical analysis. The mean thickness of inner nuclear layer (INL) and inner plexiform layer (IPL) was measured using Axiovision3.1 software (Carl Zeiss MicroImaging, Inc., Thornwood, N.Y.). Total surviving cells in the retinal ganglion cell layer at the same locations were counted using light microscopy.

Statistical analysis: Twelve fields per retina were analyzed for each treatment, and a total of 72 fields were used. For statistical analysis, one-way analysis of variance (ANOVA) and Fisher tests were performed; p<0.05 was taken as significant. Data are presented as means and standard deviations.

The results of the experiments presented are now described.

### Experimental Example 1

### Transient Ischemia Reperfusion Injury

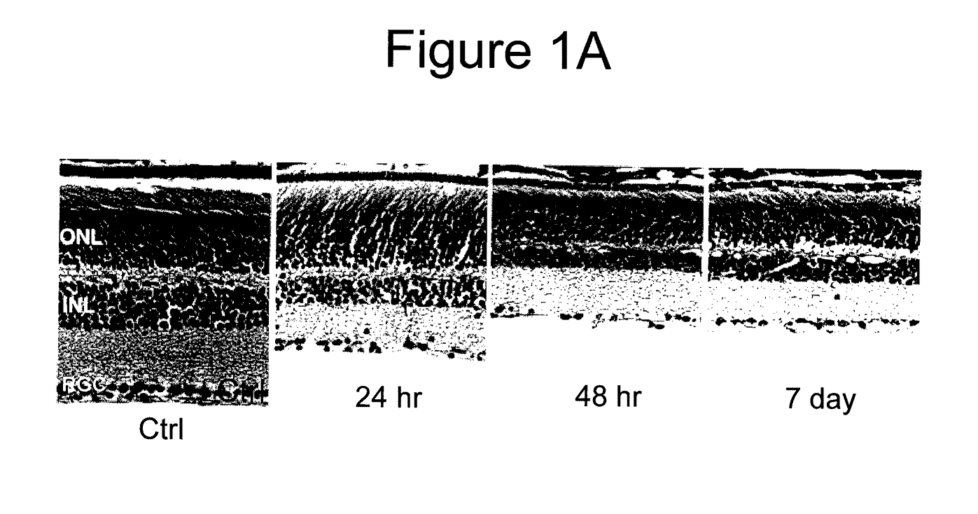

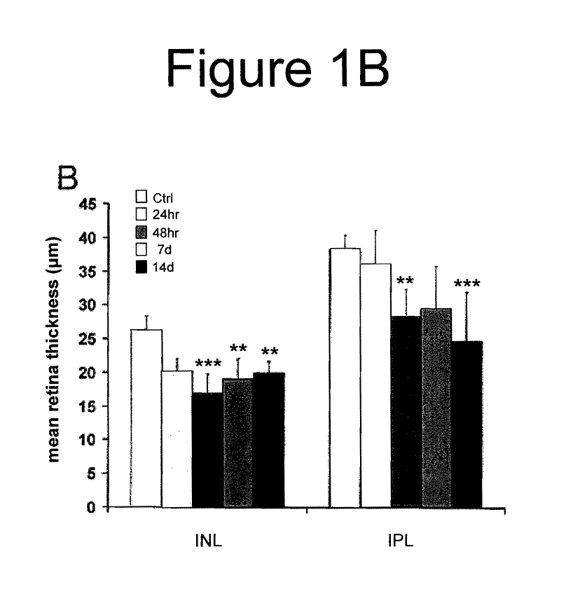

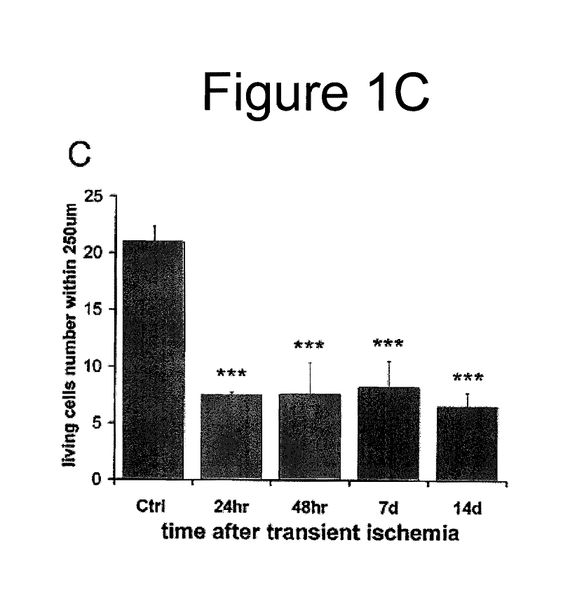

A series of experiments was carried out to measure the extent and time course of retinal damage using the ischemia-reperfusion protocol. C57/B16 mice exposed to ischemia-reperfusion showed rapid retinal damage by 24 hours and then only little additional change over the next two weeks. Compared to normal retinas (n=9), the thicknesses of inner nuclear layer (INL) in ischemic retinas were reduced to 77.0% at 24 hours (n=5), 64.1% at 48 hours (n=10), 72.6% at 7 days (n=6) and 75.9% at 14 days (n=7) (p value was versus to control group, p<0.01 at all time points) after reperfusion. (FIG. 1B, left side) A similar degree of injury was detected by measuring the changes in thickness of the inner plexiform layer (IPL), which were 93.6%, 73.6% (p<0.01), 76.5% (p<0.05) and 64.2% (p<0.001) of control respectively at the same time points (FIG. 1B, right side). The percentage of surviving cells in the retinal ganglion cell layer showed a more dramatic change and were reduced to 35.9%, 36.2%, 39.2% and 31.4% (p<0.001 in all the time points) respectively (FIG. 1C). TUNEL-positive cells in the RGC and INL layers were first observed at 12 hours and remained detectable until 48 hours after injury. Thus, as expected, this ischemia model gives a degree of general retinal damage but the most dramatic loss of cells occurs in the retinal ganglion cell layer.

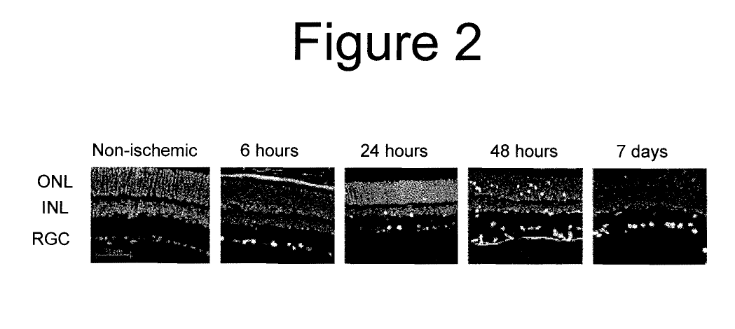

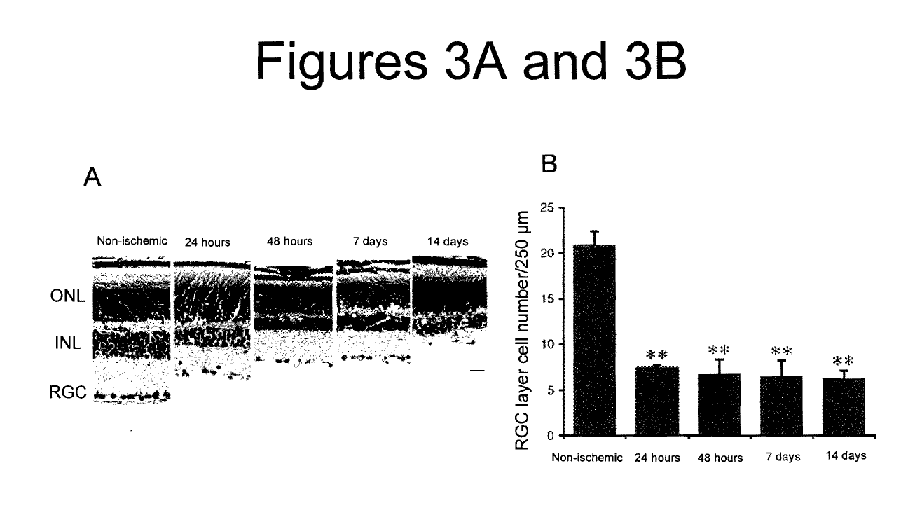

In a series of related experiments, it was observed that retinal damage occurred rapidly in C57/B16 mice, with TUNEL positive cells in the RGC layer found as early as 6 hrs after reperfusion (FIG. 2). Death of RGCs peaked at 24 hours and declined by 48 hours, consistent with trends detected by others. TUNEL positive cells were detected in the INL and ONL at 24 hours and 48 hours, respectively. Morphometric analysis shows that the percentage of surviving RGCs was 35.9%, 32.2%, 31.1% and 29.7% compared to uninjured normal retinas (p<0.001 at all time points) at 24 hours, 48 hours, 7 days, and 14 days, respectively (FIG. 3B). In addition, there was evidence of cell death in the INL by 24 hours and death in the ONL by 48 hours after injury (FIG. 3A).

### Experimental Example 2

### Full Length PEDF Protein Protects RGCs from Ischemic Injury

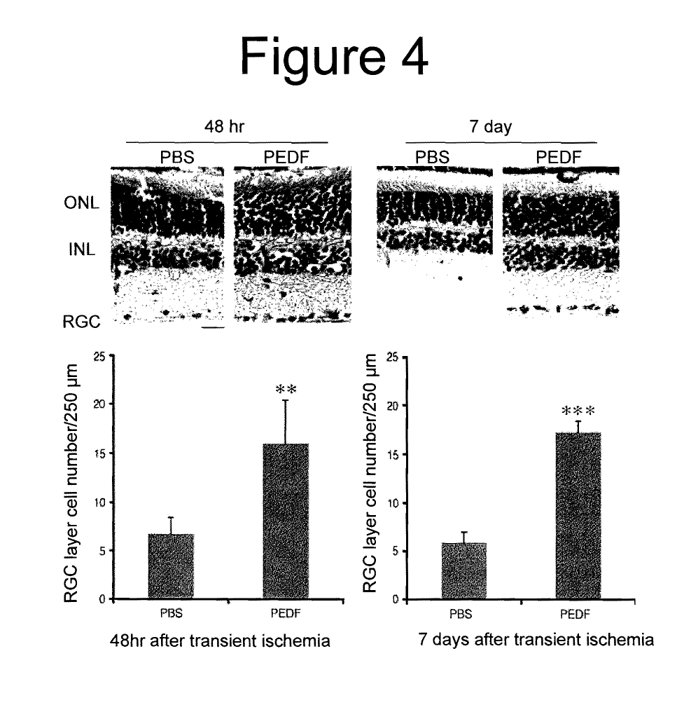

The survival of cells in the ganglion cell layer after intraocular injections with PBS (control) or purified PEDF at the time of ischemic injury was assessed. The data show that PEDF protein significantly reduced cell death in the ganglion cell layer at both 48 hours and 7 days post ischemia (FIG. 4). The protective effects of PEDF extended to other retinal layers and prevented the thinning of the IPL that normally occurs after ischemic injury.

### Experimental Example 3

### Effect of PEDF Protein and PEDF₈₂₋₁₂₁ on Ischemic Injury

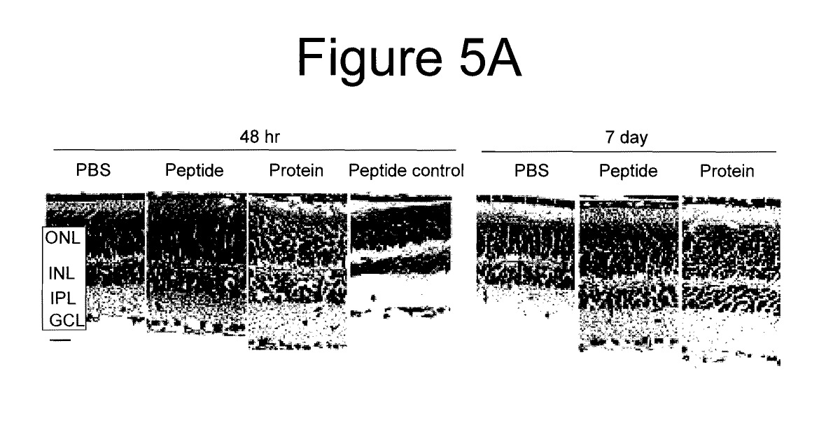

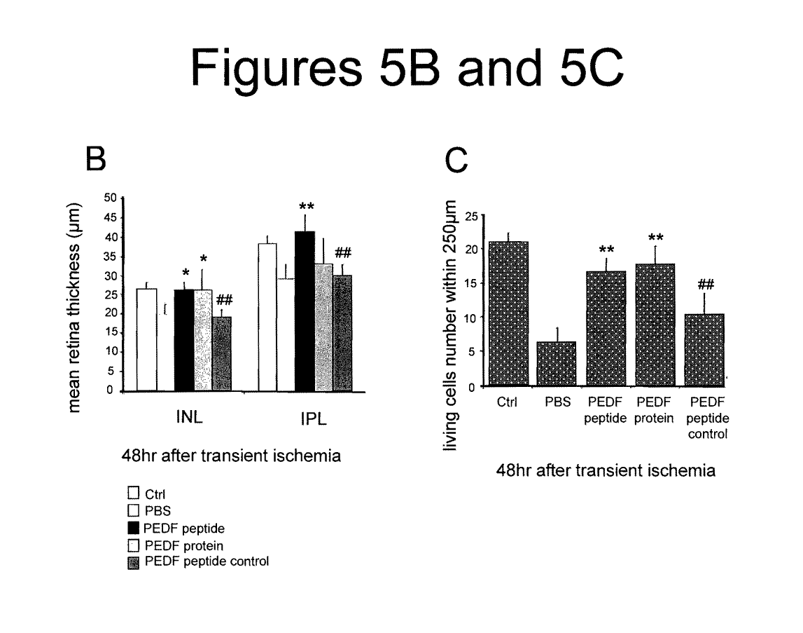

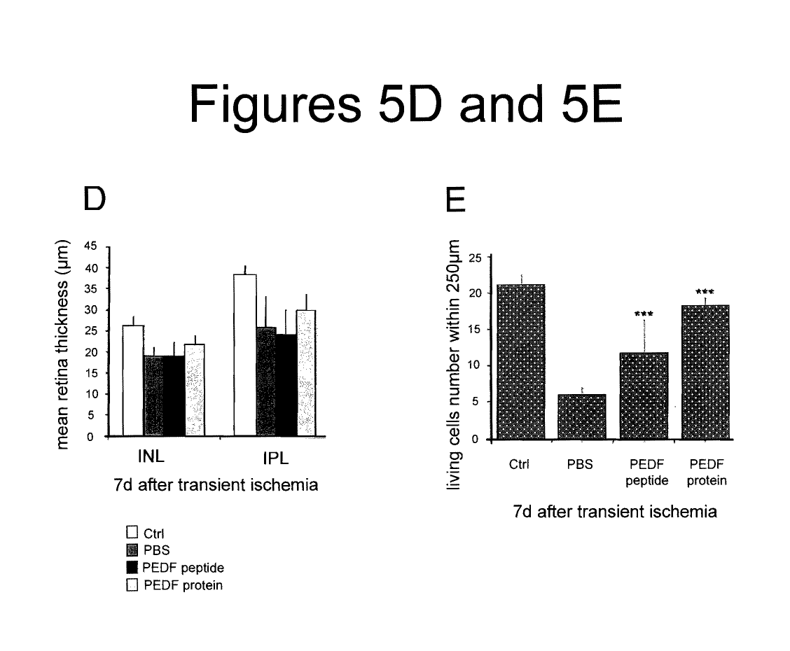

Experiments were performed to test whether a peptide fragment of PEDF, PEDF₈₂₋₁₂₁, could protect retinal cells to the same extent as PEDF using a single bolus injection at the time of injury. When compared with the non-ischemia control group, at 48 hours after ischemia reperfusion insult, the thickness of the INL, the thickness of the IPL and surviving cells in the RGC layer were reduced to 75.3%, 76.7% and 30.6% respectively in the PBS injected control group (n=6) and 72.6%, 78.1% and 50.7% in the control peptide injected control group (n=5). In the PEDF₈₂₋₁₂₁ injected group (n=6), the same parameters were 100% (p<0.05), 100% (p<0.01), 80.1% (p<0.01) of controls respectively, and in PEDF protein injected group (n=6), they were 100% (p<0.05), 86.6% and 85.4% (p<0.01) of controls respectively (p value was versus to PBS injected control group or control peptide injected group) (FIGS. 5A-5C). Thus, both PEDF₈₂₋₁₂₁ and PEDF gave substantial protection as compared to either PBS or a control peptide.

Seven (7) days after injury, INL, IPL thickness and surviving cells in the RGC layer were reduced to 72.4%, 66.8% and 28.2% in PBS injected group (n=6). In the PEDF₈₂₋₁₂₁ injected group (n=6), they were 72.8%, 62.7%, 56.8% (p<0.01) of controls respectively, and in the PEDF protein injected group (n=6), they were 82.5%, 78.2% and 87.4% (p<0.001) of controls respectively (p value was versus to PBS injected control group) (FIGS. 5A, 5D and 5E). The results indicate that both the PEDF protein and PEDF₈₂₋₁₂₁ peptide can rescue retinas exposed to ischemia-reperfusion injury.

The effect of PEDF₈₂₋₁₂₁ on STAT3 and Erk after ischemic injury was examined by Western blot. In control retinas subjected to ischemic injury, phosphorylated STAT3 was detected at 24 hours but not at 1 hour, 4 hours, 12 hours or 48 hours after ischemic injury. Administration of PEDF₈₂₋₁₂₁ sustained the presence of phosphorylated STAT3 such that it was also detected at 48 hours after ischemic injury. Furthermore, Erk phosphorylation was sustained in retinas administered PEDF₈₂₋₁₂₁. In control retinas, phosphorylated Erk was detect strongly at 4 hours and 12 hours after ischemic injury but was barely detectable at 24 hours and not detected at 48 hours. Remarkably, administration of PEDF₈₂₋₁₂₁ after ischemic injury phosphorylated Erk was strongly detected at 48 hours. Without wishing to be bound by theory, it is believed that part of the mechanism by which PEDF₈₂₋₁₂₁ protects retinal ganglion cells and other inner retinal layers important for RGC function is the continued presence of phosphorylated STAT3 and Erk.

### Experimental Example 4

### Effect of PEDF₈₂₋₁₂₁ and PEDF₁₃₆₋₁₅₅ on Ischemic Injury

The efficacy of two PEDF fragments, PEDF₈₂₋₁₂₁ and PEDF₁₃₆₋₁₅₅ on ischemic injury was compared. PEDF₈₂₋₁₂₁ is conserved in phylogeny and unique to PEDF when compared to all other proteins including other serpins, the family with which PEDF shares significant homology. PEDF₁₃₆₋₁₅₅ is a region of the protein containing a putative nuclear localization signal (NLS) (Tombran-Tink et al., 2005, J Struct Biol. 151:130-150).

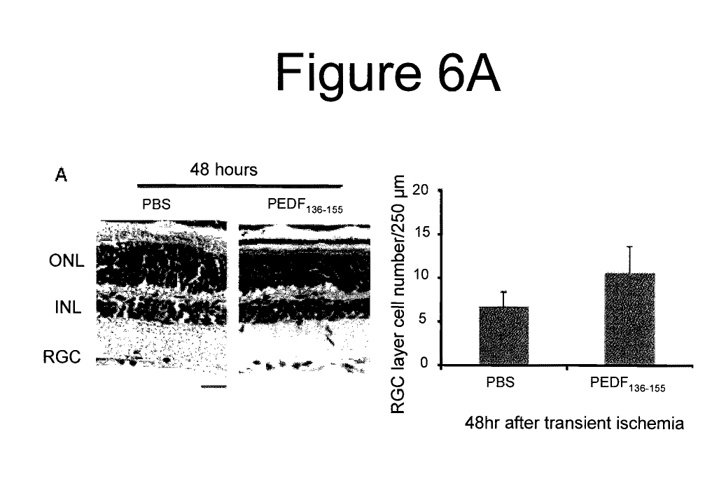

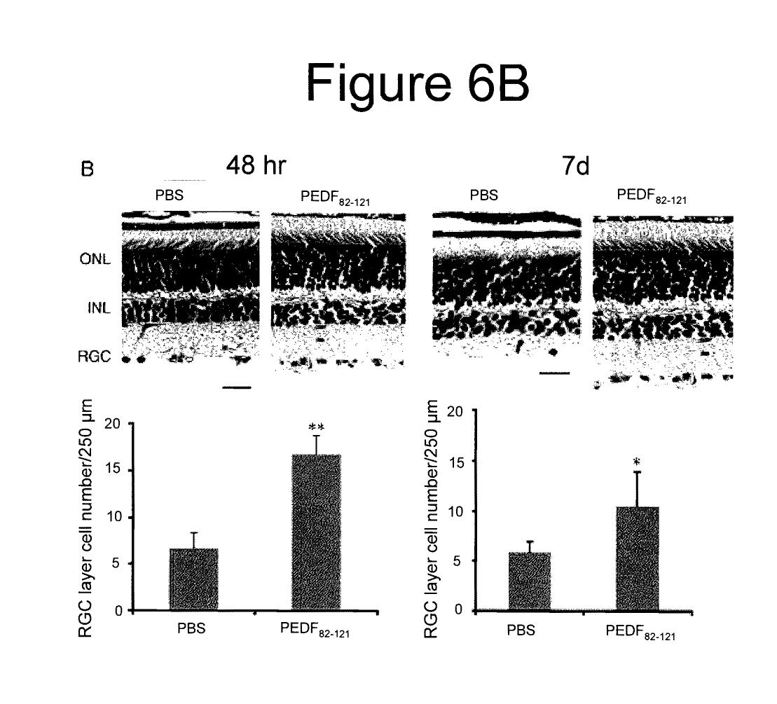

Of the two peptides fragments tested, only PEDF₈₂₋₁₂₁ was capable of protecting the retinal ganglion cell layer to the same extent as PEDF when injected as a free, single bolus. PEDF₈₂₋₁₂₁ resulted in substantial protection at 48 hr when compared to injections of PBS or PEDF₁₃₆₋₁₅₅ (FIGS. 6A and 6B). It was also observed that, like the full length PEDF protein, PEDF₈₂₋₁₂₁ maintained the thickness of the IPL compared to the PBS injected controls (FIG. 6B). By 7 days, however, the effect of the PEDF₈₂₋₁₂₁ peptide was less. Without wishing to be bound by theory, it is thought that the peptide is cleared from the retina by 7 days.

### Experimental Example 5

### Effect of Various PEDF Fragments on Ischemic Injury

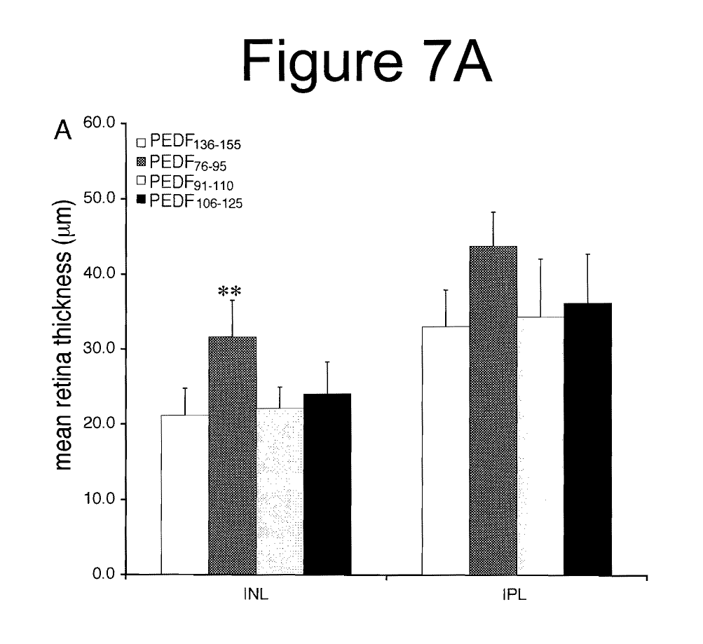

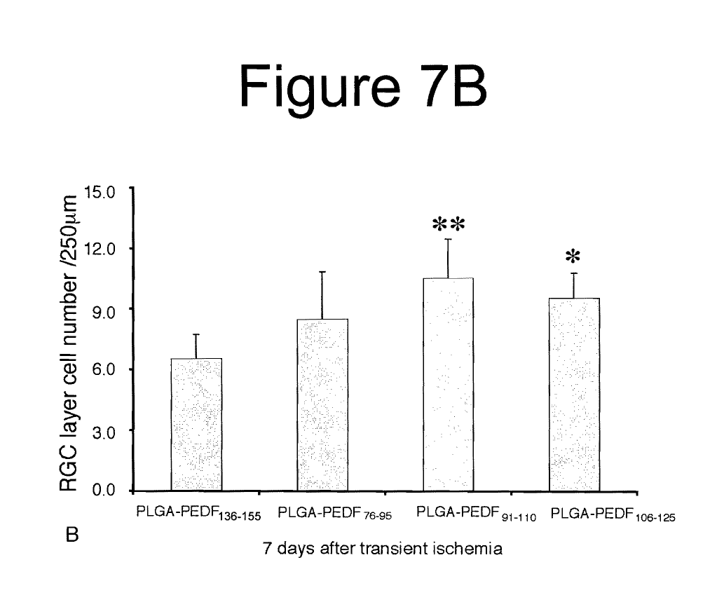

The efficacy of several addition PEDF fragments on ischemic injury were assessed using the same procedure. These fragments were: PEDF₇₆₋₉₅, PEDF₉₁₋₁₁₀ and PEDF₁₀₆₋₁₂₅. As illustrated in FIG. 7A, PEDF₇₆₋₉₅ protected the INL significantly and the IPL of retinas seven days after ischemic injury. Furthermore, both PEDF₉₁₋₁₁₀ and PEDF₁₀₆₋₁₂₅ were capable of significantly protecting the retinal ganglion cell layer when injected as a free, single bolus after ischemic injury (FIG. 7B).

### Experimental Example 6

### Release Kinetics Profile of PEDF₈₂₋₁₂₁ from PLGA Nanospheres In Vitro

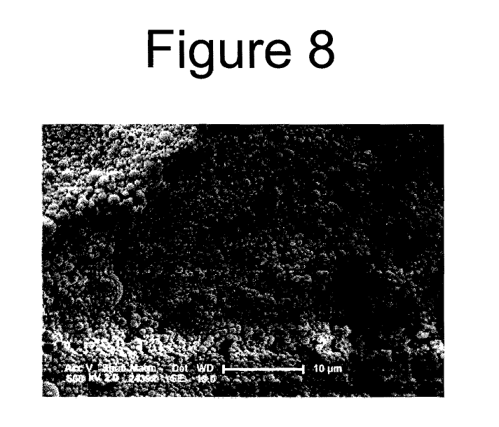

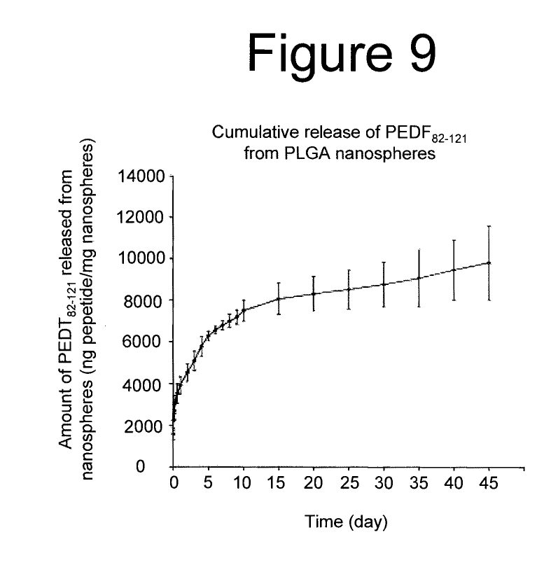

Experiments were then performed to test if there was greater protection to the inner retina after injury with injections of PLGA encapsulated PEDF₈₂₋₁₂₁ over free PEDF₈₂₋₁₂₁ peptide. These particles were mostly spherical structures with smooth surfaces and with an average size of approximately 300 nm, as shown in the scanning electron photomicrograph in FIG. 8. Almost all of the peptide was encapsulated in the nanospheres since free peptide aggregates were not found in the preparations. An in vitro controlled release experiment was carried out to determine how much of the encapsulated peptide was released over a period of time. The data indicate that 10,000 ng of encapsulated PEDF₈₂₋₁₂₁ was cumulatively released from 1 mg of nanospheres over a 45-day period (FIG. 9). Most of the peptide (75%) was released within the first 10 days, after which time the rate decreased significantly resulting in a plateauing of the cumulative release curve.

### Experimental Example 7

### Effects of PLGA Encapsulated PEDF₈₂₋₁₂₁ on Ischemia Retinas

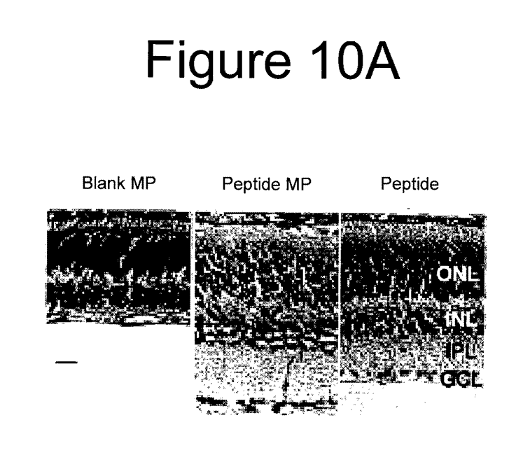

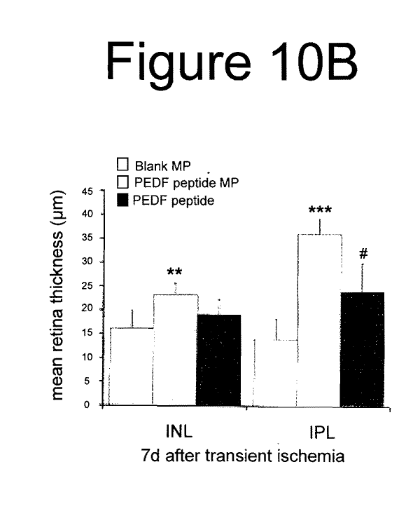

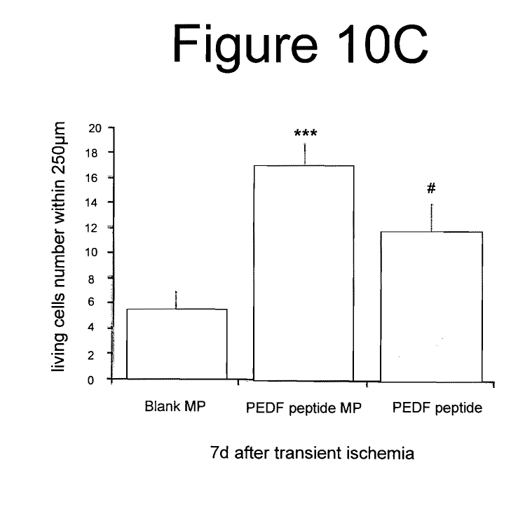

When compared with non-ischemia control eyes, 7 d after ischemia reperfusion insult, the thicknesses of the INL, IPL thickness and surviving cells in retinal ganglion layer were 61.6%, 36.6% and 26.3% respectively in the blank nanosphere injected group; while in the PEDF₈₂₋₁₂₁ nanosphere injected group (n=5) the corresponding values were 88.2% (p<0.01), 94.1% (p<0.001) and 81.3% (p<0.001) respectively (p value is versus blank nanosphere injected group). The results indicate that PEDF₈₂₋₁₂₁ has a strong rescue effect on inner retina ischemic damage, and that encapsulated PEDF₈₂₋₁₂₁ has a stronger effect than same amount of free PEDF₈₂₋₁₂₁ (FIGS. 10A-10C).

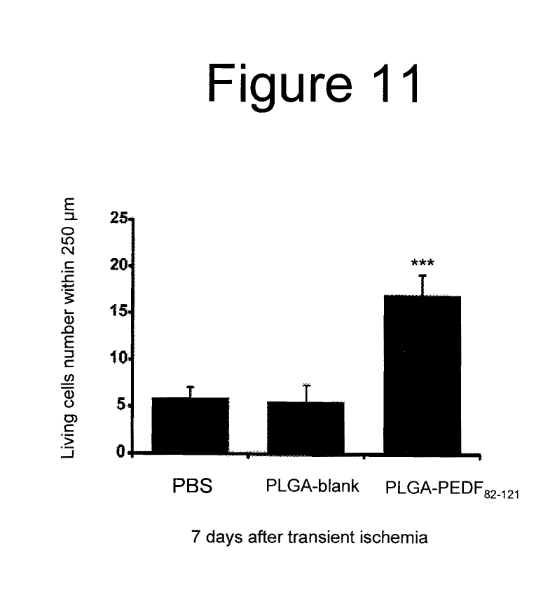

Injection of blank nanospheres into the ischemic eyes did not prevent ganglion cell death any better than injections with PBS. Neither induced a detectable level of inflammatory response, indicating that the PLGA particles themselves were relatively inert in this model. The encapsulated form of PEDF₈₂₋₁₂₁ had a strong rescue effect on ischemic damage to the RGC layer. Furthermore, PLGA-PEDF₈₂₋₁₂₁ was more effective at preserving cells in the RGC layer than the free peptide 7 days after ischemia reperfusion insult (FIG. 11).

### Experimental Example 8

### Effects of Transient Ischemia and PEDF on Retinal Glial Cells

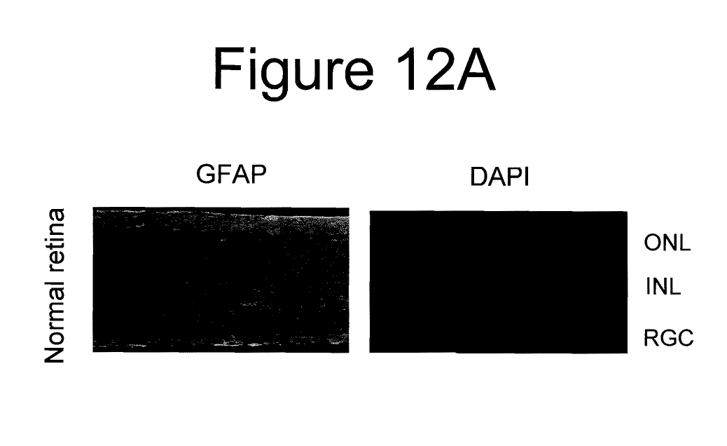

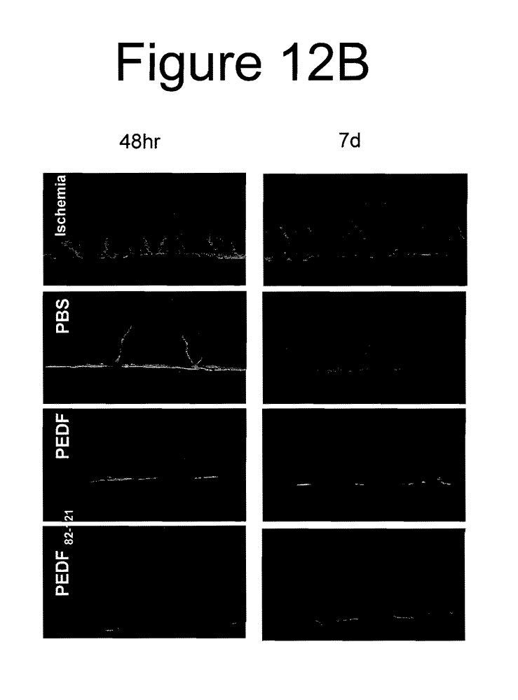

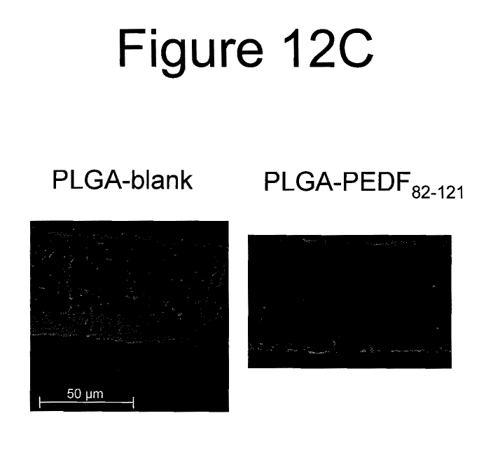

Molecular changes in Müller glial cells almost invariably accompany retinal injury and serves as a useful indicator of general retina injury. One of these changes is the increase in levels of detectable GFAP. In an analysis of ischemic injured retinas, a time-dependent increase in the expression of GFAP was observed, as detected immunocytochemically in the end feet and fibers of the Müller cells that span the whole retina (FIGS. 12A and 12B). At 48 hours after transient ischemia, retinas injected with PEDF₈₂₋₁₂₁ or PEDF protein had less GFAP labeling when compared to PBS injections. This effect was transient since all three groups had very similar staining at 7 days after injury.

Comparison of sections from eyes injected with blank nanospheres or PEDF peptide encapsulated nanospheres at the time of ischemic injury exhibited that at 7 days, there was GFAP staining in both groups but that the sections from animals injected with blank nanospheres showed much stronger staining (FIG. 12C). From the results of this series of experiments, it is concluded that PEDF and PEDF₈₂₋₁₂₁ peptide not only protect the inner retina from damage but also delay the glial response to ischemic injury.

### Experimental Example 9

### Codon-Optimized Full-Length PEDF

Bacterial expression of human full-length PEDF is problematic. Bacterial growth is inhibited when recombinant human PEDF is expressed. In addition, the expressed PEDF is subject to protealytic degradation. To address this issues, a sequence encoding human PEDF was codon-optimized for expression in E. coli. The codon-optimized sequence is provided in SEQ ID NO: 11. A total of 318 nucleotides were altered, thus changing about 255 of the nucleotides. The amino acid sequence of the encoded protein (SEQ ID NO: 12) is unchanged from a naturally-occurring PEDF (GenBank® Accession No. M76979).

The codon-optimized sequence was cloned into pET-32a (Novagen, Madison, Wis.) between the unique KpnI and BamHI sites. The resultant sequence encodes a fusion protein comprising thioredoxin (a 109 amino acid protein) fused via intervening elements including His₆ tag, a thrombin cleavage site and S•Tag™ (Novagen, Madison Wis.) to the N-terminal of human PEDF.

The codon-optimized fusion protein (coPEDF) was expressed in E. coli BL-21(DE3) as follows. Expression of the codon-optimized fusion PEDF was induced with 0.5 mmol/liter IPTG for 5 hours at 25° C. Cells were harvested by centrifugation. The fusion protein was purified by resuspending the bacterial pellet in a phosphate buffer. The cells were disrupted by sonication on ice. The sonicated cells were centrifuged at high speed for 20 minutes and the supernatant discarded. The pellet, containing inclusion bodies comprising coPEDF was washed twice in 2 M urea, 1% Triton X-100, 50 mM Tris-HCL buffer, pH 8.0. The precipitate was resolved in 20 ml of 8 M urea, 2 mM DDT, 50 mM Tris-HCL buffer, pH 8.0, and then centrifuged at high speed to remove the insoluble part. The supernatant was collected, diluted in 100 ml of 50 mM Tris-HCL buffer, pH 8.0 and dialyzed overnight against 20 mM Tris-HCL buffer, pH 8.0 to permit refolding of proteins. The dialysate was loaded onto a Ni-IDA column (Genscript, Piscataway, N.J.) to affinity purify the refolded coPEDF comprising His₆.

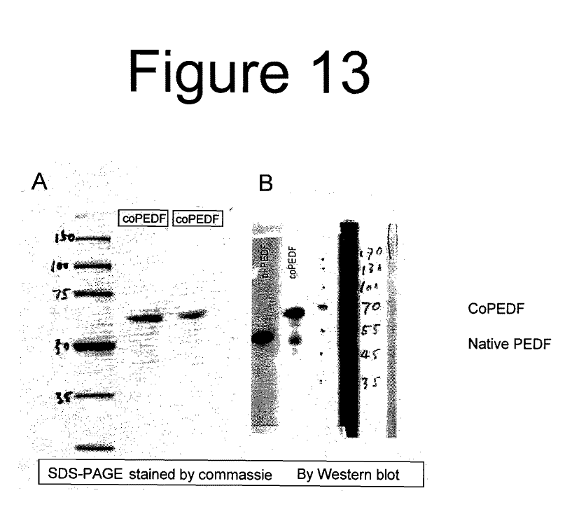

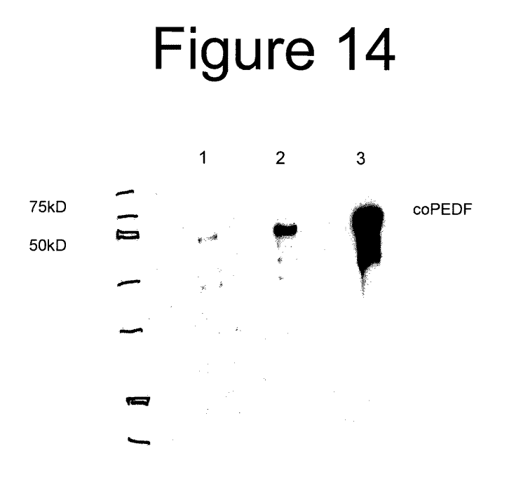

The purified coPEDF was electrophoresed using SDS-PAGE and a Western blot was performed, using a polyclonal antibody against PEDF made by conventional techniques. As shown in FIG. 13, coPEDF migrated slightly slower than PEDF, as expected. FIG. 14 depicts a Western blot of increasing amounts of coPEDF, exhibiting the low level of degradation observed in expressing the codon-optimized coPEDF fusion protein in bacterial. Protein expression concentration of coPEDF was about 22 mg/liter (3 grams of cells) in contrast to about 1.5 mg/liter (3 grams of cells; Becerra et al., 1993, J Biol Chem. 268:23148-23156). Thus, protein expression and subsequent yield of codon-optimized coPEDG was significantly higher than prior art results for bacterially-expressed human PEDF.

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A peptide consisting essentially of a sequence selected from the group consisting of residues 82 to 121 of a human pigment epithelium-derived factor (PEDF), residues 76 to 95 of a human PEDF, residues 91 to 110 of a human PEDF, residues 106 to 125 of a human PEDF and variants thereof.

2. The peptide of claim 1, wherein said sequence comprises residues 82 to 121 of human PEDF or a variant thereof.

3. The peptide of claim 1, wherein said sequence comprises residues 76 to 95 of human PEDF, or a variant thereof.

4. The peptide of claim 1, wherein said sequence comprises residues 91-110 of human PEDF, or a variant thereof.

5. A composition comprising a peptide consisting essentially of a sequence selected from the group consisting of residues 82 to 121 of a human pigment epithelium-derived factor (PEDF), residues 76 to 95 of a human PEDF, residues 91 to 110 of a human PEDF, residues 106 to 125 of a human PEDF and variants thereof, in a pharmaceutically acceptable carrier.

6. A fusion protein comprising a first peptide operably fused to a second peptide, wherein said first peptide consists essentially of a sequence selected from the group consisting of residues 82 to 121 of a human pigment epithelium-derived factor (PEDF), residues 76 to 95 of a human PEDF, residues 91 to 110 of a human PED, residues 106 to 125 of a human PEDF F and variants thereof, wherein said second peptide is not a PEDF peptide.

7. The fusion protein of claim 6, wherein said second peptide is selected from the group consisting of His6, FLAG-tag, Myc, LacZ, a cellular targeting sequence and a therapeutic polypeptide.

8. A composition comprising a fusion protein comprising a first peptide operably fused to a second peptide, and a pharmaceutically acceptable carrier, wherein said first peptide consists essentially of a sequence selected from the group consisting of residues 82 to 121 of a human pigment epithelium-derived factor (PEDF), residues 76 to 95 of a human PEDF, residues 91 to 110 of a human PEDF, residues 106 to 125 of a human PEDF and variants thereof, wherein said second peptide is not a PEDF peptide.

9. The composition of claim 8, wherein said second peptide is selected from the group consisting of His6, FLAG-tag, Myc, LacZ, a cellular targeting sequence and a therapeutic polypeptide.

10. A fusion protein comprising a first peptide operably fused to a second peptide, wherein said first peptide consists essentially of a sequence selected from the group consisting of residues 82 to 121 of a human pigment epithelium-derived factor (PEDF), residues 76 to 95 of a human PEDF, residues 91 to 110 of a human PEDF, residues 106 to 125 of a human PEDF and variants thereof, and said second peptide is a PEDF peptide that consists essentially of a sequence selected from the group consisting of residues 82 to 121 of a human pigment epithelium-derived factor (PEDF), residues 76 to 95 of a human PEDF, residues 91 to 110 of a human PEDF, residues 106 to 125 of a human PEDF and variants thereof, wherein said first and second peptides are not identical to each other.

11. A composition comprising a fusion protein comprising a first peptide operably fused to a second peptide, and a pharmaceutically acceptable carrier, wherein said first peptide consists essentially of a sequence selected from the group consisting of residues 82 to 121 of a human pigment epithelium-derived factor (PEDF), residues 76 to 95 of a human PEDF, residues 91 to 110 of a human PEDF, residues 106 to 125 of a human PEDF and variants thereof, and said second peptide is a PEDF peptide that consists essentially of a sequence selected from the group consisting of residues 82 to 121 of a human pigment epithelium-derived factor (PEDF), residues 76 to 95 of a human PEDF, residues 91 to 110 of a human PEDF, residues 106 to 125 of a human PEDF and variants thereof, wherein said first and second peptides are not identical to each other.

12. The peptide of claim 1, wherein said peptide is encapsulated in a biocompatible delivery polymer.

13. The peptide of claim 12, wherein said biocompatible delivery polymer is selected from the group consisting of a hydrogel, a polymer comprising gelatin, a polymer comprising collagen, a polymer comprising alginate, and a polymer comprising poly (lactide-co-glycolide) (PGLA).

14. The peptide of claim 13, wherein said biocompatible delivery polymer is poly (lactide-co-glycolide) (PGLA).

15. The peptide of claim 12, wherein said peptide is contained within a nanotube or a microsphere.

16. The composition of claim 5, wherein said peptide is encapsulated in a biocompatible delivery polymer.

17. The composition of claim 16, wherein said peptide is contained within a nanotube or a microsphere.

18. The fusion protein of claim 6, wherein said fusion protein is encapsulated in a biocompatible delivery polymer.

19. The fusion protein of claim 18, wherein said fusion protein is contained within a nanotube or a microsphere.

20. The composition of claim 8, wherein said fusion protein is encapsulated in a biocompatible delivery polymer.

21. The composition of claim 20, wherein said fusion protein is contained within a nanotube or a microsphere.

22. The fusion protein of claim 10, wherein said fusion protein is encapsulated in a biocompatible delivery polymer.

23. The fusion protein of claim 22, wherein said fusion protein is contained within a nanotube or a microsphere.

24. The composition of claim 11, wherein said fusion protein is encapsulated in a biocompatible delivery polymer.

25. The composition of claim 24, wherein said fusion protein is contained within a nanotube or a microsphere.

26. A kit comprising the peptide of claim 1, and an instructional material for the use therefor.

27. A kit comprising the composition of claim 5, and an instructional material for the use therefor.

28. A kit comprising the fusion protein of claim 6, and an instructional material for the use therefor.

29. A kit comprising the composition of claim 8, and an instructional material for the use therefore.

30. A kit comprising the fusion protein of claim 10, and an instructional material for the use therefore.

31. A kit comprising the composition of claim 11, and an instructional material for the use therefore.

32. An isolated nucleic acid encoding a peptide consisting essentially of a sequence selected from the group consisting of residues 82 to 121 of a human pigment epithelium-derived factor (PEDF), residues 76 to 95 of a human PEDF, residues 91 to 110 of a human PEDF, residues 106 to 125 of a human PEDF and variants thereof.

33. A vector comprising the isolated nucleic acid of claim 32.

34. A cell comprising the isolated nucleic acid of claim 32.

35. An isolated nucleic acid encoding a fusion protein comprising a first peptide operably fused to a second peptide, wherein said first peptide consists essentially of a sequence selected from the group consisting of residues 82 to 121 of a human pigment epithelium-derived factor (PEDF), residues 76 to 95 of a human PEDF, residues 91 to 110 of a human PEDF, residues 106 to 125 of a human PEDF and variants thereof, wherein said second peptide is not a PEDF peptide.

36. A vector comprising the isolated nucleic acid of claim 35.

37. A cell comprising the isolated nucleic acid of claim 35.

38. An isolated nucleic acid encoding a fusion protein comprising a first peptide operably fused to a second peptide, wherein said first peptide consists essentially of a sequence selected from the group consisting of residues 82 to 121 of a human pigment epithelium-derived factor (PEDF), residues 76 to 95 of a human PEDF, residues 91 to 110 of a human PEDF, residues 106 to 125 of a human PEDF and variants thereof, and said second peptide is a PEDF peptide that consists essentially of a sequence selected from the group consisting of residues 82 to 121 of a human pigment epithelium-derived factor (PEDF), residues 76 to 95 of a human PEDF, residues 91 to 110 of a human PEDF, residues 106 to 125 of a human PEDF and variants thereof, wherein said first and second peptides are not identical to each other.

39. A vector comprising the isolated nucleic acid of claim 38.

40. A cell comprising the isolated nucleic acid of claim 38.

41. A method of treating a neurodegenerative disease in a mammal, said method comprising administering to said mammal a therapeutically effective amount of a peptide consisting essentially of a sequence selected from the group consisting of residues 82 to 121 of a human pigment epithelium-derived factor (PEDF), residues 76 to 95 of a human PEDF, residues 91 to 110 of a human PEDF, residues 106 to 125 of a human PEDF and variants thereof.

42. The method of claim 41, wherein said mammal is a human.

43. The method of claim 41, wherein said neurodegenerative disease is selected from the group consisting of Parkinson's disease, Huntington's disease, Alzheimer's disease, diabetic retinopathy, and an ocular disease.

44. The method of claim 43, wherein said neurodegenerative disease is an ocular disease.

45. The method of claim 44, wherein said ocular disease is selected from the group consisting of: ischemic injury, diabetic retinopathy, retinopathy of prematurity, macular degeneration and glaucoma.

46. A method of treating a neurodegenerative disease in a mammal, said method comprising administering to said mammal a therapeutically effective amount of a fusion protein comprising a first peptide operably fused to a second peptide, wherein said first peptide consists essentially of a sequence selected from the group consisting of residues 82 to 121 of a human pigment epithelium-derived factor (PEDF), residues 76 to 95 of a human PEDF, residues 91 to 110 of a human PEDF, residues 106 to 125 of a human PEDF and variants thereof, wherein said second peptide is not a PEDF peptide.

47. The method of claim 46, wherein said mammal is a human.

48. A method of treating a neurodegenerative disease in a mammal, said method comprising administering to said mammal a therapeutically effective amount of a fusion protein comprising a first peptide operably fused to a second peptide, wherein said first peptide consists essentially of a sequence selected from the group consisting of residues 82 to 121 of a human pigment epithelium-derived factor (PEDF), residues 76 to 95 of a human PEDF, residues 91 to 110 of a human PEDF, residues 106 to 125 of a human PEDF and variants thereof, and said second peptide is a PEDF peptide that consists essentially of a sequence selected from the group consisting of residues 82 to 121 of a human pigment epithelium-derived factor (PEDF), residues 76 to 95 of a human PEDF, residues 91 to 110 of a human PEDF, residues 106 to 125 of a human PEDF and variants thereof, wherein said first and second peptides are not identical to each other.

49. A method of treating a neurodegenerative disease in a mammal, said method comprising administering to said mammal a therapeutically effective amount of an isolated nucleic acid encoding a peptide consisting essentially of a sequence selected from the group consisting of residues 82 to 121 of a human pigment epithelium-derived factor (PEDF), residues 76 to 95 of a human PEDF, residues 91 to 110 of a human PEDF, residues 106 to 125 of a human PEDF and variants thereof.

50. A method of treating a neurodegenerative disease in a mammal, said method comprising administering to said mammal a therapeutically effective amount of an isolated nucleic acid encoding a fusion protein comprising a first peptide operably fused to a second peptide, wherein said first peptide consists essentially of a sequence selected from the group consisting of residues 82 to 121 of a human pigment epithelium-derived factor (PEDF), residues 76 to 95 of a human PEDF, residues 91 to 110 of a human PEDF, residues 106 to 125 of a human PEDF and variants thereof, wherein said second peptide is not a PEDF peptide.

51. A method of treating a neurodegenerative disease in a mammal, said method comprising administering to said mammal a therapeutically effective amount of an isolated nucleic acid encoding a fusion protein comprising a first peptide operably fused to a second peptide, wherein said first peptide consists essentially of a sequence selected from the group consisting of residues 82 to 121 of a human pigment epithelium-derived factor (PEDF), residues 76 to 95 of a human PEDF, residues 91 to 110 of a human PEDF, residues 106 to 125 of a human PEDF and variants thereof, and said second peptide is a PEDF peptide that consists essentially of a sequence selected from the group consisting of residues 82 to 121 of a human pigment epithelium-derived factor (PEDF), residues 76 to 95 of a human PEDF, residues 91 to 110 of a human PEDF, residues 106 to 125 of a human PEDF and variants thereof, wherein said first and second peptides are not identical to each other.

52. An isolated nucleic acid comprising SEQ ID NO: 11 or variants thereof.

53. A vector comprising the isolated nucleic acid of claim 52.

54. The vector of claim 53, further comprising one or more of a thioredoxin coding sequence and a His₆ coding sequence operably linked to SEQ ID NO: 11 or variants thereof.

55. A cell comprising the isolated nucleic acid of claim 52.

56. A method of preparing human PEDF in a bacterial cell, said method comprising:
a) introducing an expression cassette comprising SEQ ID NO: 11 or variants thereof into a bacterium;
b) expressing said expression cassette to produce a human PEDF protein; and
c) purifying said human PEDF protein.

57. The peptide of claim 1, wherein said sequence comprises residues 106-125 of human PEDF, or a variant thereof.
